(19) United States
(12) Patent Application Publication
(10) Pub. No.: US 2018/0008672 A1
(43) Pub. Date: Jan. 11, 2018

(54) ANTI-ACTIVIN A ANTIBODIES AND METHODS OF USE THEREOF FOR TREATING PULMONARY ARTERIAL HYPERTENSION

(71) Applicant: Regeneron Pharmaceuticals, Inc., Tarrytown, NY (US)

(72) Inventors: Dan Chalothorn, New York, NY (US); Lori C. Morton, Chappaqua, NY (US)

(21) Appl. No.: 15/643,539

(22) Filed: Jul. 7, 2017

Related U.S. Application Data
(60) Provisional application No. 62/453,600, filed on Feb. 2, 2017.
(60) Provisional application No. 62/359,840, filed on Jul. 8, 2016.

(51) Int. Cl. A61K 38/18; C07K 16/28; A61K 39/00; C07K 14/495; C12N 15/09

(52) U.S. Cl. CPC A61K 38/1841; C07K 14/495; C12N 15/09; A61K 39/00; C07K 16/2863; C07K 2319/32; C07K 2319/30

(57) ABSTRACT

The present invention provides anti-Activin A antibodies, and antigen-binding fragments thereof, as well as methods of use of such antibodies, or antigen-binding fragments thereof, for treating a subject having pulmonary arterial hypertension (PAH).

### RELATED APPLICATIONS

This application claims the benefit of priority to U.S. Provisional Application No. 62/359,840, filed on Jul. 8, 2016, and U.S. Provisional Application No. 62/453,600, filed on Feb. 2, 2017. The entire contents of each of the foregoing applications is hereby incorporated herein by reference.

### SEQUENCE LISTING

The instant application contains a Sequence Listing which has been submitted electronically in ASCII format and is hereby incorporated by reference in its entirety. Said ASCII copy, created on Jul. 5, 2017, is named 10264US01_SeqListing.txt and is 87,396 bytes in size.

### BACKGROUND OF THE INVENTION

Pulmonary arterial hypertension (PAH) is a progressive disorder characterized by a sustained increase in pulmonary artery pressure which damages both the large and small pulmonary arteries. PAH is defined hemodynamically as a systolic pulmonary artery pressure greater than 30 mm Hg or evaluation of mean pulmonary artery pressure greater than 25 mm Hg with a pulmonary capillary or left atrial pressure equal to or less than 15 mm Hg. See, e.g., Zaiman et al., Am. J. Respir. Cell Mol. Biol. 33:425-31 (2005). The persistent vasoconstriction in PAH leads to structural remodeling during which pulmonary vascular smooth muscle cells and endothelial cells undergo a phenotypic switch from a contractile normal phenotype to a synthetic phenotype leading to cell growth and matrix deposition. As the walls of the smallest blood vessels thicken, they are less able to transfer oxygen and carbon dioxide normally between the blood and the lungs and, in time, pulmonary hypertension leads to thickening of the pulmonary arteries and narrowing of the passageways through which blood flows. Eventually, the proliferation of vascular smooth muscle and endothelial cells leads to remodeling of the vessels with obliteration of the lumen of the pulmonary vasculature. Histological examination of tissue samples from patients with pulmonary hypertension shows intimal thickening, as well as smooth muscle cell hypertrophy, especially for those vessels <100 μm diameter. This causes a progressive rise in pulmonary pressures as blood is pumped through decreased lumen area. As a consequence, the right side of the heart works harder to compensate and the increased effort causes the right ventricle to become enlarged and thickened. The enlarged right ventricle places a person at risk for pulmonary embolism because blood tends to pool in the ventricle and in the legs. If clots form in the pooled blood, they may eventually travel and lodge in the lungs. Eventually, the additional workload placed on the right ventricle causes the heart to fail and leads to premature death in these patients.

Standard therapies for treatment of subjects having PAH are primarily hemodynamic, influencing vessel tone and include, e.g., prostacyclin analogs, endothelin receptor antagonists, phosphodiesterase inhibitors and soluble guanylate cyclases activators/stimulators, which provide symptomatic relief and improve prognosis. However, these therapies fall short and do not re-establish the structural and functional integrity of the lung vasculature to provide a patient having PAH with handicap-free long-term survival.

There are many cellular pathways that could lead to the development of PAH and the structural remodeling in PAH such as, for example, the transforming growth factor-beta (TGF-β) pathway and/or bone morphogenic protein (BMP) pathway (see FIG. 1). A pathogenic role for members of the TGF-β superfamily in PAH has been suggested by the discovery that mutations in genes encoding the TGF-β receptor superfamily proteins BMPR2, ACVRL1, or ENG, or the signal transducer, SMAD9, which increase a person's susceptibility to heritable forms of PAH. It has also been shown that PAH patients have reduced BMPR2 expression/signaling (Atkinson et al. Circulation. 105(14):1672-1678, 2002; Alastalo et al. J. Clin. Invest. 121:3735-3746, 2011), that TGF-β activation of pulmonary artery smooth muscle cells is insensitive to growth inhibition with loss of BMPR2 (Morrell et al. Circulation. 104(7):790-7952001; Yang et al. Circ. Res. 102, 1212-1221, 2008), and that BMP9 activation of BMPR2 reverses preclinical PAH (Long et al. Nat Med. 21: 777-785, 2015). Furthermore, Activin A has been shown to significantly enhance proliferation of human pulmonary artery smooth muscle cells and to be elevated in serum and lungs of patients with pulmonary arterial hypertension. In addition, in mouse models of pulmonary hypertension, Activin A expression was elevated and associated with increased pulmonary vascular remodeling (Yndestad et al. 2009).

Activins, members of the transforming growth factor-beta (TGF-β) superfamily, are homo- or heterodimers of InhibinβA, InhibinβB, InhibinβC and InhibinβE, and different combinations of these dimers create the various members of the activin protein group. For example, Activin A is a homodimer of InhibinβA and Activin B is a homodimer of InhibinβB, whereas Activin AB is a heterodimer of InhibinβA and InhibinβB and Activin AC is a heterodimer of InhibinβA and InhibinβC (Tsuchida, K. et al., Cell Commun Signal 7:15 (2009)).

Activin A binds to and activates receptor complexes on the surface of cells known as Activin Type II receptors (Type IIA and Type IIB, also known as ActRIIA and ActRIIB, respectively). The activation of these receptors leads to the phosphorylation of an Activin Type I receptor (e.g., Alk4 or Alk7), which in turn leads to the phosphorylation of SMAD 2 and 3 proteins, the formation of SMAD complexes (with SMAD4), and the translocation of the SMAD complex to the cell nucleus, where SMAD2 and SMAD3 function to regulate transcription of various genes (Sozzani, S. and Musso, T., Blood 117(19):5013-5015 (2011)) (FIG. 1; (Villapol, et al. (2013) in Trends in Cell Signaling Pathways in Neuronal Fate Decision. ed. Wislet-Gendebien, S. DOI: 10.5772/3445).). Follistatin regulates activin A bioactivity by preventing activin A/receptor interaction (Chen YG, et al. Exp Biol Med (Maywood) 227: 75-87, 2002).

Despite all the advances in the therapy of PAH there is as yet no prospect of cure of this deadly disease and the majority of patients continue to progress to right ventricular failure. Thus, there is a need in the art for clinically beneficial methods and compositions that target vascular remodeling regulated by the TGFβ and BMP pathways to decrease TGFβ signaling and increase BMP signaling by inhibiting Activin A.

### SUMMARY OF THE INVENTION

The present invention is based, at least in part, on the discovery that anti-Activin A antibodies, or antigen-binding fragments thereof, are effective for ameliorating the effects of vascular remodeling in animal models of pulmonary arterial hypertension.

Accordingly, in one aspect, the present invention provided methods for treating a subject having pulmonary arterial hypertension (PAH). The methods include administering to the subject a therapeutically effective amount of an anti-Activin A antibody, or antigen-binding fragment thereof, wherein administration of the anti-Activin A antibody, or antigen-binding fragment thereof, to the subject inhibits thickening of the pulmonary artery in the subject, thereby treating the subject having PAH.

In another aspect, the present invention provides methods of treating a subject having pulmonary arterial hypertension (PAH). The methods include administering to the subject a therapeutically effective amount of an anti-Activin A antibody, or antigen-binding fragment thereof, wherein administration of the anti-Activin A antibody, or antigen-binding fragment thereof, to the subject increases stroke volume in the subject, thereby treating the subject having PAH.

In yet another aspect, the present invention provides methods of treating a subject having pulmonary arterial hypertension (PAH). The methods include administering to the subject a therapeutically effective amount of an anti-Activin A antibody, or antigen-binding fragment thereof, wherein administration of the anti-Activin A antibody, or antigen-binding fragment thereof, to the subject increases right ventricle cardiac output in the subject, thereby treating the subject having PAH.

In another aspect, the present invention provides methods of treating a subject having pulmonary arterial hypertension (PAH). The methods include administering to the subject a therapeutically effective amount of an anti-Activin A antibody, or antigen-binding fragment thereof, wherein administration of the anti-Activin A antibody, or antigen-binding fragment thereof, to the subject increases survival time, thereby treating the subject having PAH.

In one embodiment, the subject is human.

In one embodiment, the subject has Group I (WHO) PAH.

The methods of the invention may further include administering to the subject at least one additional therapeutic agent, such as an anticoagulant, a diuretic, a cardiac glycoside, a calcium channel blocker, a vasodilator, a prostacyclin analogue, an endothelium antagonist, a phosphodiesterase inhibitor, an endopeptidase inhibitor, a lipid lowering agent, and/or a thromboxane inhibitor.

Antibodies, or antigen-binding fragments thereof, for use in the present invention may specifically bind Activin A with a binding dissociation equilibrium constant (K_{D}) of less than about 5 pM as measured in a surface plasmon resonance assay at 25° C., or may specifically bind Activin A with a binding dissociation equilibrium constant (K_{D}) of less than about 4 pM as measured in a surface plasmon resonance assay at 25° C.

In one embodiment, antibodies, or antigen-binding fragments thereof, for use in the present invention specifically bind Activin A with a binding association equilibrium constant (Kₐ) of less than about 500 nM.

In another embodiment, antibodies, or antigen-binding fragments thereof, for use in the present invention block binding of at least one Activin A receptor to Activin A.

In yet another embodiment, antibodies, or antigen-binding fragments thereof, for use in the present invention block activation of at least one Activin A receptor by Activin A.

In one embodiment, antibodies, or antigen-binding fragments thereof, for use in the present invention do not significantly block binding of Activin A to an Activin Type II receptor.

Antibodies, or antigen-binding fragments thereof, for use in the present invention may block Activin A binding to an Activin A receptor with an IC₅₀ value of less than about 80 pM as measured in an in vivo receptor/ligand binding bioassay at 25° C., or may block Activin A binding to an Activin A receptor with an IC₅₀ value of less than about 60 pM as measured in an in vivo receptor/ligand binding bioassay at 25° C.

In one embodiment, antibodies, or antigen-binding fragments thereof, for use in the present invention inhibit binding of Activin A to an Activin A receptor selected from the group consisting of Activin Type IIA receptor (ActRIIA), Activin Type IIB receptor (ActRIIB), and Activin Type I receptor.

In another embodiment, antibodies, or antigen-binding fragments thereof, for use in the present invention inhibit Activin A-mediated activation of SMAD complex signaling.

In one embodiment, antibodies, or antigen-binding fragments thereof, for use in the present invention compete for binding to Activin A with a reference antibody comprising a heavy chain variable region (HCVR)/light chain variable region (LCVR) sequence pair selected from the group consisting of SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

In another embodiment, antibodies, or antigen-binding fragments thereof, for use in the present invention bind to the same epitope on Activin A as a reference antibody comprising an HCVR/LCVR sequence pair selected from the group consisting of SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

In one embodiment, antibodies, or antigen-binding fragments thereof, for use in the present invention comprise the complementarity determining regions (CDRs) of a HCVR having an amino acid sequence selected from the group consisting of SEQ ID NOs: 2, 18, 34, 50, 66, 82, 98, 106, 114, 122, 130, 138, 154, 162, 170, 178, 186, 194, and 202; and the CDRs of a LCVR having an amino acid sequence selected from the group consisting of SEQ ID NOs: 10, 26, 42, 58, 74, 90, 146, and 210.

In another embodiment, antibodies, or antigen-binding fragments thereof, for use in the present invention comprise the heavy and light chain CDRs of a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210, e.g., the antibodies, or antigen-binding fragments thereof, comprise HCDR1-HCDR2-HCDR3-LCDR1-LCDR2-LCDR3 domains, respectively, selected from the group consisting of: SEQ ID NOs: 4-6-8-12-14-16; 20-22-24-28-30-32; 36-38-40-44-46-48; 52-54-56-60-62-64; 68-70-72-76-78-80; 84-86-88-92-94-96; 100-102-104-92-94-96; 108-110-112-92-94-96; 116-118-120-92-94-96; 124-126-128-92-94-96; 132-134-136-92-94-96; 140-142-144-148-150-152; 156-158-160-148-150-152; 164-166-168-148-150-152; 172-174-176-148-150-152; 180-182-184-148-150-152; 188-190-192-148-150-152; 196-198-200-148-150-152; and 204-206-208-212-214-216.

In yet another embodiment, antibodies, or antigen-binding fragments thereof, for use in the present invention comprise a HCVR having an amino acid sequence selected from the group consisting of SEQ ID NOs: 2, 18, 34, 50, 66, 82, 98, 106, 114, 122, 130, 138, 154, 162, 170, 178, 186, 194, and 202; and a LCVR having an amino acid sequence selected from the group consisting of SEQ ID NOs: 10, 26, 42, 58, 74, 90, 146, and 210, e.g., the antibodies or antigen-binding fragments thereof, comprise a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

### BRIEF DESCRIPTION OF THE DRAWINGS

FIG. 1 schematically depicts cellular signaling through the transforming growth factor-beta (TGFβ) pathway and the bone morphogenic protein (BMP) pathway, as well as the crosstalk between the two pathways (Villapol, et al. (2013) in Trends in Cell Signaling Pathways in Neuronal Fate Decision. ed. Wislet-Gendebien, S. DOI: 10.5772/3445).

FIG. 2A is a graph depicting the effect of administration of REGN2477 on pulmonary artery (PA) cross-sectional area (CSA) in a chronic hypoxia mouse model of pulmonary arterial hypertension.

FIG. 2B is a graph depicting the effect of administration of REGN2477 on right ventricular stroke volume in a chronic hypoxia mouse model of pulmonary arterial hypertension.

FIG. 2C is a graph depicting the effect of administration of REGN2477 on right ventricular hypertrophy calculated as the weight of the right ventricle (RV) divided by the weight of the left ventricle (LV)+the weight of the septum (S) in a chronic hypoxia mouse model of pulmonary arterial hypertension.

FIG. 2D is a graph depicting the effect of administration of REGN2477 on right ventricular systolic pressure in a chronic hypoxia mouse model of pulmonary arterial hypertension.

FIG. 3A is a graph depicting the effect of administration of H4H10430P or H4H10446P2 on pulmonary artery (PA) cross-sectional area (CSA) in a rat model of pulmonary arterial hypertension induced by monocrotaline administration.

FIG. 3B is a graph depicting the effect of administration of REGN2477 on right ventricular stroke volume in a rat model of pulmonary arterial hypertension induced by monocrotaline administration.

FIG. 3C is a graph depicting the effect of administration of REGN2477 on right ventricle hypertrophy calculated as the weight of the right ventricle (RV) divided by the weight of the left ventricle (LV)+the weight of the septum (S) in a rat model of pulmonary arterial hypertension induced by monocrotaline administration.

FIG. 3D is a graph depicting the effect of administration of REGN2477 on right ventricular systolic pressure in a rat model of pulmonary arterial hypertension induced by monocrotaline administration.

FIG. 4 is a graph depicting the effect of administration of REGN2477 on survival in a rat model of pulmonary arterial hypertension induced by monocrotaline administration.

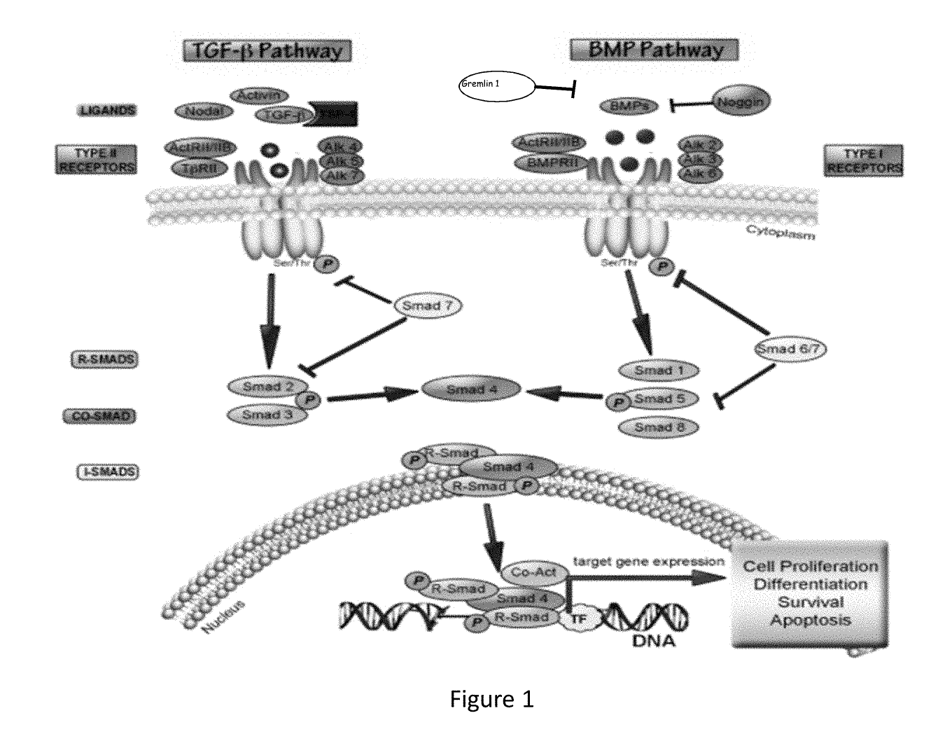

### DETAILED DESCRIPTION OF THE INVENTION

The present invention is based, at least in part, on the discovery that anti-Activin A antibodies, or antigen-binding fragments thereof, are effective for ameliorating the effects of vascular remodeling in animal models of pulmonary arterial hypertension. The following detailed description discloses how to make and use compositions containing anti-Activin A antibodies, or antigen-binding fragments thereof, to selectively inhibit the activity of Activin A as well as compositions, uses, and methods for treating subjects having pulmonary arterial hypertension (PAH).

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, ranges include both the upper and lower limit.

The term “pulmonary hypertension” (“PH”) is a term used to describe high blood pressure in the lungs from any cause. The terms “hypertension” or “high blood pressure,” on the other hand, refer to high blood pressure in the arteries throughout the body.

The term “pulmonary arterial hypertension” (“PAH”) refers to a progressive lung disorder which is characterized by sustained elevation of pulmonary artery pressure. Those patients with PAH typically have pulmonary artery pressure that is equal to or greater than 25 mm Hg with a pulmonary capillary or left atrial pressure equal to or less than 15 mm Hg. These pressures are typically measured in a subject at rest using right-heart catheterization. PAH, when untreated, leads to death (on average) within 2.8 years after being diagnosed.

The World Health Organization (WHO) has provided a clinical classification of PAH of five groups (Simonneau, et al. J Am Coll Cardiol. 2013;62 (25_S), the entire contents of which are incorporated herein by reference):
1. Pulmonary arterial hypertension (PAH)
   1.1. Idiopathic
   1.2. Heritable
      1.2.1. BMPR2
      1.2.2. ALK1, ENG, SMAD9, CAV1, KCNK3
      1.2.3. Unknown
   1.3. Drug- and toxin-induced
   1.4. Associated with:
      1.4.1. Connective tissue diseases
      1.4.2. HIV infection
      1.4.3. Portal Hypertension
      1.4.4. Congenital heart diseases
      1.4.5. Schistosomiasis
1′. Pulmonary veno-occlusive disease (PVOD) and/or pulmonary capillary hemangiomatosis (PCH)
1″. Persistent pulmonary hypertension of the newborn (PPHN)
2. Pulmonary hypertension due to left heart disease
   2.1. Left ventricular systolic dysfunction
   2.2. Left ventricular diastolic dysfunction
   2.3. Valvular disease
   2.4. Congenital/acquired left heart inflow/outflow tract obstruction and congenital cardiomyopathies
3. Pulmonary hypertension due to lung disease and/or hypoxia
   3.1. Chronic obstructive pulmonary disease
   3.2. Interstitial lung disease
   3.3. Other pulmonary diseases with mixed restrictive and obstructive pattern
   3.4. Sleep-disordered breathing
   3.5. Alveolar hypoventilation disorders
   3.6. Chronic exposure to high altitude
   3.7. Developmental abnormalities
4. Chronic thromboembolic pulmonary hypertension (CTEPH)
5. Pulmonary hypertension with unclear multifactorial mechanisms
   5.1. Hematologic disorders: chronic hemolytic anemia, myeloproliferative disorders, splenectomy
   5.2. Systemic disorders: sarcoidosis, pulmonary histiocytosis, lymphangioleimoyomatosis
   5.3. Metabolic disorders: glycogen storage disease, Gaucher disease, thyroid disorders
   5.4. Others: tumoral obstruction, fibrosing mediastinitis, chronic renal failure on dialysis, segmental PH.

In one embodiment, a subject that would benefit from the methods of the present invention is a subject having Group I (WHO) PAH.

PAH at baseline (e.g., when diagnosed) can be mild, moderate or severe, as measured, for example, by the WHO functional class, which is a measure of disease severity in patients with PAH. The WHO functional classification is an adaptation of the New York Heart Association (NYHA) system and is routinely used to qualitatively assess activity tolerance, for example, in monitoring disease progression and response to treatment (Rubin (2004) Chest 126:7-10). There are four functional classes recognized in the WHO system:

Class I: pulmonary hypertension without resulting limitation of physical activity; ordinary physical activity does not cause undue dyspnea or fatigue, chest pain or near syncope;

Class II: pulmonary hypertension resulting in slight limitation of physical activity; patient comfortable at rest; ordinary physical activity causes undue dyspnea or fatigue, chest pain or near syncope;

Class III: pulmonary hypertension resulting in marked limitation of physical activity; patient comfortable at rest; less than ordinary activity causes undue dyspnea or fatigue, chest pain or near syncope; and

Class IV: pulmonary hypertension resulting in inability to carry out any physical activity without symptoms; patient manifests signs of right-heart failure; dyspnea and/or fatigue may be present even at rest; discomfort is increased by any physical activity.

In one embodiment, a subject that would benefit from the methods of the present invention is a subject having, at baseline, PAH e.g., Group I (WHO) PAH) of WHO Class I. In another embodiment, a subject that would benefit from the methods of the present invention is a subject having, at baseline, PAH (e.g., Group I (WHO) PAH) of WHO Class II. In another embodiment, a subject that would benefit from the methods of the present invention is a subject having, at baseline, PAH e.g., Group I (WHO) PAH) of WHO Class III.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

In one embodiment, the subject is a human, such as a human being treated or assessed for PAH e.g., Group I (WHO) PAH; a human at risk for PAH e.g., Group I (WHO) PAH; a human having PAH e.g., Group I (WHO) PAH; and/or human being treated for PAH e.g., Group I (WHO) PA), as described herein.

As used herein, the terms “treating” or “treatment” refer to a beneficial or desired result including, but not limited to, alleviation or amelioration of one or more symptoms associated with PAH e.g., Group I (WHO) PAH). “Treatment” can also mean slowing the course of the disease or reducing the development of a symptom of disease, reducing the severity of later-developing disease, or prolonging survival as compared to expected survival in the absence of treatment. For example, the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective treatment. “Therapeutically effective amount,” as used herein, is intended to include the amount of an anti-Activin A antibody, or antigen-binding fragment thereof, that, when administered to a subject having PAH e.g., Group I (WHO) PAH, is sufficient to effect treatment of the disease (e.g., by diminishing, ameliorating or maintaining the existing disease or one or more symptoms of disease) or manage the disease. The “therapeutically effective amount” may vary depending on the anti-Activin A antibody, or antigen-binding fragment thereof, how the anti-Activin A antibody, or antigen-binding fragment thereof, is administered, the disease and its severity and the history, age, weight, family history, genetic makeup, stage of PAH, the types of preceding or concomitant treatments, if any, and other individual characteristics of the patient to be treated.

A “therapeutically effective amount” is also intended to include the amount of an anti-Activin A antibody, or antigen-binding fragment thereof, that, when administered to a subject is sufficient to ameliorate the disease or one or more symptoms of the disease. Ameliorating the disease includes slowing the course of the disease or reducing the severity of later-developing disease.

A “therapeutically-effective amount” also includes an amount of an anti-Activin A antibody, or antigen-binding fragment thereof, that produces some desired local or systemic effect at a reasonable benefit/risk ratio applicable to any treatment. Anti-Activin A antibodies, or antigen-binding fragments thereof, employed in the methods of the present invention may be administered in a sufficient amount to produce a reasonable benefit/risk ratio applicable to such treatment.

### II. Methods of the Invention

The present invention provides methods for treating a subject having pulmonary arterial hypertension. The methods generally include administering to the subject a therapeutically effective amount of an anti-Activin A antibody, or antigen-binding fragment thereof.

In some aspects of the present invention, administration of the anti-Activin A antibody, or antigen-binding fragment thereof, inhibits thickening of the pulmonary artery in the subject, e.g., inhibit further thickening of the pulmonary artery in the subject from baseline, e.g., at diagnosis. The thickening of the pulmonary artery may be determined by, for example, chest CT (such as, unenhanced axial 10 mm CT sections), and used to calculate main pulmonary artery diameter (mPA). The main pulmonary artery diameter in normal subjects is about 2.4 cm to about 3.0 cm. Main pulmonary artery diameter in subjects with pulmonary arterial hypertension is about 3.1 cm to about 3.8 cm, or greater. See, e.g., Edwards, et al. (1998) Br J Radiol 71(850):1018-20.

In other aspects of the present invention, administration of the anti-Activin A antibody, or antigen-binding fragment thereof, increases stroke volume and/or stroke volume to end systolic volume ratio (“SV/ESV”) in the subject. “Stroke volume” (“SV”) is the volume of blood pumped from the right or left ventricle per single contraction. Stroke volume may be calculated using measurements of ventricle volumes from an echocardiogram and calculated by subtracting the volume of the blood in the ventricle at the end of a beat (called “end-systolic volume,” “EDV”) from the volume of blood just prior to the beat (called “end-diastolic volume,” “ESV”). Stroke volume may also be calculated, e.g., as cardiac out put measured by thermodilution during right heart catheterization divided by heart rate or as EDV minus ESV and indexed for body surface area. The term stroke volume can apply to each of the two ventricles of the heart. The stroke volumes for each ventricle are generally equal, both being approximately 70 mL in a healthy subjects. The SV/ESV for healthy subjects is about 0.9 to about 2.2 and the SV/ESV for subjects having PAH is about 0.2 to about 0.9. See, e.g. Brewis, et al. (2016) Int J Cardiol 218:206-211.

In yet other aspects of the present invention, administration of the anti-Activin A antibody, or antigen-binding fragment thereof, increases right ventricle cardiac output and/or cardiac index (CI) in the subject. “Cardiac output” (“CO”) is defined as the amount of blood pumped by a ventricle in unit time. “Cardiac index” (“CI”) is a haemodynamic parameter that relates the cardiac output (CO) from left ventricle in one minute to “body surface area” (“BSA”), thus relating heart performance to the size of the individual. Echocardiographic techniques and radionuclide imaging techniques can be used to estimate real-time changes in ventricular dimensions, thus computing stroke volume, which when multiplied by heart rate, gives cardiac output, and BSA may be calculated using any one of the formulas known to one of ordinary skill in the art including, for example, the Du Bois formula Verbraecken, J, et al. (2006) Metabolism-Clin Exper 55(4):515-24) or the Mosteller formula (Mosteller (1987) N Engl J Med 317:1098). Subjects that do not have PAH have a cardiac output in the range of about 4.0-8.0 L/min and a cardiac index of about 2.6 to about 4.2 L/minute per square meter. Subjects that have PAH have a cardiac index of about 1.9 to about 2.3 L/minute per square meter (Ryan and Archer (2016) Circ Res 115:176-188).

In other aspects of the present invention, administration of the anti-Activin A antibody, or antigen-binding fragment thereof, increases survival time of the subject. For example, the methods of the present invention may prolong the life of a subject having PAH from a time of initiation of treatment by, for example, at least about 15 days, at least about 30 days, at least about 60 days, at least about 90 days, at least about 120 days, at least about 150 days, at least about 180 days, at least about 210 days, at least about 240 days, at least about 270 days, about least about 300 days, at least about 330 days, at least about 360 days, at least about 1.5 years, at least about 2 years, at least about 2.5 years, at least about 3 years, at least about 3.5 years, at least about 4 years, at least about 4.5 years, or at least about 5 years.

Administration of the anti-Activin A antibody, or antigen-binding fragment thereof, to a subject having PAH in the methods of the present invention may improve other hemodynamic measurements in a subject having PAH, such as, for example, right atrium pressure, pulmonary artery pressure, pulmonary capillary wedge pressure in the presence of end expiratory pressure, systemic artery pressure, heart beat, pulmonary vascular resistance, and/or systemic vascular resistance. Methods and devices for measuring right atrium pressure, pulmonary artery pressure, pulmonary capillary wedge pressure in the presence of end expiratory pressure, systemic artery pressure, heart beat, pulmonary vascular resistance, and/or systemic vascular resistance are known to one of ordinary skill in the art.

Subjects that do not have PAH have a right atrium pressure of about 1 mm Hg to about 5 mm Hg; subjects that have PAH have a right atrium pressure of about 11 mm Hg to about 13 mm Hg.

Subjects that do not have PAH have a pulmonary artery pressure of about 9 mm Hg to about 20 mm Hg; subjects that have PAH have a pulmonary artery pressure of about 57 mm Hg to about 61 mm Hg.

Subjects that do not have PAH have a pulmonary capillary wedge pressure in the presence of end expiratory pressure of about 4 mm Hg to about 12 mm Hg; subjects that have PAH have a pulmonary capillary wedge pressure in the presence of end expiratory pressure of about 9 mm Hg to about 11 mm Hg.

Subjects that do not have PAH have a systemic artery pressure of about 90 mm Hg to about 96 mm Hg; subjects that have PAH have a systemic artery pressure of about 87 mm Hg to about 91 mm Hg.

Subjects that do not have PAH have a heart beat of about 60 beats per minute (bpm) to about 90 bpm; subjects that have PAH have a systemic artery pressure of about 84 bpm 88 bpm.

Subjects that do not have PAH have a pulmonary vascular resistance of about 20 dynes s/cm⁵ to about 130 dynes s/cm⁵ (or about 0.25 to about 1.625 wood units) subjects that have PAH have a pulmonary vascular resistance of about 1200 dynes s/cm⁵ to about 1360 dynes s/cm⁵ (or about 15 to about 17 wood units).

Subjects that do not have PAH have a systemic vascular resistance of about 700 dynes s/cm⁵ to about 1600 dynes s/cm⁵ (or about 9 to about 20 wood units) subjects that have PAH have a systemic vascular resistance of about 1840 dynes s/cm⁵ to about 2000 dynes s/cm⁵ (or about 23 to about 25 wood units).

The methods of the present invention may also improve other clinical parameters, such as pulmonary function, in the subject being treated. For example, during or following a treatment period a subject may have an increased exercise capacity or activity, as measured by, for example, a test of 6-minute walking distance (6 MWD) or measure of activity, or lowering Borg dyspnea index (BDI).

The methods of the present invention may also improve one or more quality of life parameters versus baseline, for example an increase in score on at least one of the SF-36® health survey functional scales; an improvement versus baseline in the severity of the condition, for example by movement to a lower WHO functional class; and/or an increased longevity.

Any suitable measure of exercise capacity can be used to determine whether a subject has an increased exercise capacity or activity. One suitable measure is a 6-minute walk test (6 MWT), which measures how far the subject can walk in 6 minutes, i.e., the 6-minute walk distance (6 MWD). Another suitable measure is the Borg dyspnea index (BDI) which is a numerical scale for assessing perceived dyspnea (breathing discomfort). It measures the degree of breathlessness after completion of the 6 minute walk test (6 MWT), where a BDI of 0 indicates no breathlessness and 10 indicates maximum breathlessness. In one embodiment, the methods of the invention provide to the subject an increase from baseline in the 6 MWD by at least about 10 minutes, e.g., about 10, 15, 20, or about 30 minutes. In another embodiment, following a 6 MWT the methods of the invention provide to the subject a lower from baseline BDI by at least about 0.5 to about 1.0 index points.

Any suitable measure quality of life may be used. For example, the SF-36® health survey provides a self-reporting, multi-item scale measuring eight health parameters: physical functioning, role limitations due to physical health problems, bodily pain, general health, vitality (energy and fatigue), social functioning, role limitations due to emotional problems, and mental health (psychological distress and psychological well-being). The survey also provides a physical component summary and a mental component summary. In one embodiment, the methods of the invention provide to the subject an improvement versus baseline in at least one of the SF-36 physical health related parameters (physical health, role-physical, bodily pain and/or general health) and/or in at least one of the SF-36 mental health related parameters (vitality, social functioning, role-emotional and/or mental health). Such an improvement can take the form of an increase of at least 1, for example at least 2 or at least 3 points, on the scale for any one or more parameters.

The methods of the present invention may also improve the prognosis of the subject being treated. For example, the methods of the invention may provide to the subject a reduction in probability of a clinical worsening event during the treatment period, and/or a reduction from baseline in serum brain natriuretic peptide (BNP) or NT pro-BNP or its N-terminal prohormone, NT-pro-BNP concentration, wherein, at baseline, time from first diagnosis of the condition in the subject is not greater than about 2 years.

Time from first diagnosis, in various aspects, can be, for example, not greater than about 1.5 years, not greater than about 1 year, not greater than about 0.75 year or not greater than about 0.5 year. A clinical worsening event (CWE) includes death, lung transplantation, hospitalization for the PAH, atrial septostomy, initiation of additional pulmonary hypertension therapy or a combination thereof. Time to clinical worsening of PAH is defined as the time from initiation of treatment to the first occurrence of a CWE.

In one embodiment, the methods of the invention provide a reduction from baseline of at least about 15%, for example at least about 25%, at least about 50% or at least about 75%, in BNP or NT-pro-BNP concentration.

In one embodiment, the methods of the invention provide a reduction of at least about 25%, for example at least about 50%, at least about 75%> or at least about 80%, in probability of death, lung transplantation, hospitalization for pulmonary arterial hypertension, atrial septostomy and/or initiation of additional pulmonary hypertension therapy during the treatment period.

The therapeutically effective amount of an anti-Activin A antibody, or antigen-binding fragment thereof, for use in the methods of the invention may be from about 0.05 mg to about 600 mg; e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg, of the respective antibody.

The amount of anti-Activin A antibody, or antigen-binding fragment thereof, contained within an individual dose may be expressed in terms of milligrams of antibody per kilogram of patient body weight (i.e., mg/kg). For example, an anti-Activin A antibody, or antigen-binding fragment thereof, may be administered to a patient at a dose of about 0.0001 to about 50 mg/kg of patient body weight (e.g. 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.5 mg/kg, 18.0 mg/kg, 18.5 mg/kg, 19.0 mg/kg, 19.5 mg/kg, 20.0 mg/kg, etc.).

Multiple doses of an anti-Activin A antibody, or antigen-binding fragment thereof, or a pharmaceutical composition comprising an anti-Activin A antibody, or antigen-binding fragment thereof, may be administered to a subject over a defined time course. The methods according to this aspect of the invention comprise sequentially administering to a subject multiple doses of an active ingredient of the invention. As used herein, “sequentially administering” means that each dose of an active ingredient is administered to the subject at a different point in time, e.g., on different days separated by a predetermined interval (e.g., hours, days, weeks or months). The present invention includes methods which comprise sequentially administering to the patient a single initial dose of an active ingredient, followed by one or more secondary doses of the active ingredient, and optionally followed by one or more tertiary doses of the active ingredient.

The terms “initial dose,” “secondary doses,” and “tertiary doses,” refer to the temporal sequence of administration of an anti-Activin A antibody, or antigen-binding fragment thereof, or of a combination therapy of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-Activin A antibody, or antigen-binding fragment thereof, but may differ from one another in terms of frequency of administration. In certain embodiments, however, the amount of anti-Activin A antibody, or antigen-binding fragment thereof, contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of an anti-Activin A antibody, or antigen-binding fragment thereof, which is administered to a patient prior to the administration of the very next dose in the sequence with no intervening doses.

The methods according to this aspect of the invention may comprise administering to a patient any number of secondary and/or tertiary doses. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiment of the present invention, an anti-Activin A antibody, or antigen-binding fragment thereof, may be administered as a monotherapy (i.e., as the only therapeutic agent). In other embodiments of the present invention, an anti-Activin A antibody, or antigen-binding fragment thereof, may be administered in combination with one or more additional therapeutic agents.

In the combination methods of the invention which comprise administering an anti-Activin A antibody, or antigen-binding fragment thereof, and at least one additional therapeutic agent to the subject, the antibody and the additional therapeutic agent may be administered to the subject at the same or substantially the same time, e.g., in a single therapeutic dosage, or in two separate dosages which are administered simultaneously or within less than about 5 minutes of one another. Alternatively, the antibody and the additional therapeutic agent may be administered to the subject sequentially, e.g., in separate therapeutic dosages separated in time from one another by more than about 5 minutes.

Accordingly, in one embodiment, the methods of the invention further comprise administering a therapeutically effective amount of at least one therapeutic agent selected from the group consisting of an anticoagulant, a diuretic, a cardiac glycoside, a calcium channel blocker, a vasodilator, a prostacyclin analogue, an endothelium antagonist, a phosphodiesterase inhibitor, an endopeptidase inhibitor, a lipid lowering agent, and a thromboxane inhibitor. In one embodiment, the methods of the invention further comprise administering a therapeutically effective amount of at least one or more additional therapeutic antibody or antibodies, or antigen-binding fragment or fragments thereof. In one embodiment, the one or more additional antibody or antibodies are selected from the group consisting of an anti-Grem 1 antibody of antibodies, an anti-PDGFRβ antibody of antibodies, an anti-TLR4 antibody of antibodies, an anti-TLR2 antibody of antibodies, an anti-EDN1 antibody of antibodies, and an anti-ASIC1 antibody of antibodies.

Examples of suitable anticoagulants include, but are not limited to, e.g. warfarin useful in the treatment of patients with pulmonary hypertension having an increased risk of thrombosis and thromboembolism.

Examples of suitable calcium channel blockers include, but are not limited to, diltiazem, felodipine, amlodipine and nifedipine.

Suitable vasodilators include, but are not limited to, e.g. prostacyclin, epoprostenol, treprostinil and nitric oxide (NO).

Suitable exemplary phosphodiesterase inhibitors include, but are not limited to, particularly phospho-diesterase V inhibitors such as e.g. tadalafil, sildenafil and vardenafil.

Examples of suitable endothelin antagonists include, but are not limited to, e.g. bosentan and sitaxentan.

Suitable prostacyclin analogues include, but are not limited to, e.g. ilomedin, treprostinil and epoprostenol.

Suitable lipid lowering agents include, but are not limited to, e.g. HMG CoA reductase inhibitors such as simvastatin, pravastatin, atorvastatin, lovastatin, itavastatin, fluvastatin, pitavastatin, rosuvastatin, ZD-4522 and cerivastatin

Diuretics suitable for use in the combination therapies of the invention include, but are not limited to, e.g. chlorthalidon, indapamid, bendro-flumethiazid, metolazon, cyclopenthiazid, polythiazid, mefrusid, ximapid, chlorothiazid and hydrochlorothiazid.

Examples of other therapeutics agents include, but are not limited to, e.g. ACE inhibitors such as enalapril, ramipril, captopril, cilazapril, trandolapril, fosinopril, quinapril, moexipril, lisinopril and perindopril, or ATII inhibitors such as losartan, candesartan, irbesartan, embusartan, valsartan and telmisartan, or iloprost, betaprost, L-arginine, omapatrilat, oxygen, and/or digoxin.

The methods of the invention may also include the combined use of kinase inhibitors (e.g., BMS-354825, canertinib, erlotinib, gefitinib, imatinib, lapatinib, lestaurtinib, lonafarnib, pegaptanib, pelitinib, semaxanib, tandutinib, tipifarnib, vatalanib, lonidamine, fasudil, leflunomide, bortezomib, imatinib, erlotinib and glivec) and/or elastase inhibitors.

The additional therapeutically active component(s) may be administered to a subject prior to administration of an anti-Activin A antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component.

In other embodiments, the additional therapeutically active component(s) may be administered to a subject after administration of an anti-Activin A antibody, or antigen-binding fragment thereof. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component.

In yet other embodiments, the additional therapeutically active component(s) may be administered to a subject concurrent with administration of anti-Activin A antibody, or antigen-binding fragment thereof, of the present invention. “Concurrent” administration, for purposes of the present invention, includes, e.g., administration of an anti-Activin A antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-Activin A antibody and the additional therapeutically active component may be administered intravenously, subcutaneously, intravitreally, etc.); alternatively, each dosage form may be administered via a different route (e.g., the anti-Activin A antibody may be administered locally (e.g., intravitreally) and the additional therapeutically active component may be administered systemically). In any event, administering the components in a single dosage from, in separate dosage forms by the same route, or in separate dosage forms by different routes are all considered “concurrent administration,” for purposes of the present disclosure. For purposes of the present disclosure, administration of an anti-Activin A antibody “prior to,” “concurrent with,” or “after” (as those terms are defined herein above) administration of an additional therapeutically active component is considered administration of an anti-Activin A antibody, or antigen-binding fragment thereof, “in combination with” an additional therapeutically active component).

### III. Binding Proteins Suitable For Use in the Methods of the Invention

Suitable anti-Activin A binding proteins for use in the methods of the present invention are described in, for example, U.S. Patent Publication No. 2015/0037339, the entire contents of which are incorporated herein by reference.

In one embodiment, a binding protein suitable for use in the present invention is an antigen-specific binding protein.

As used herein, the expression “antigen-specific binding protein” means a protein comprising at least one domain which specifically binds a particular antigen. Exemplary categories of antigen-specific binding proteins include antibodies, antigen-binding portions of antibodies, peptides that specifically interact with a particular antigen (e.g., peptibodies), receptor molecules that specifically interact with a particular antigen, and proteins comprising a ligand-binding portion of a receptor that specifically binds a particular antigen.

The present invention includes antigen-specific binding proteins that specifically bind Activin A, i.e., “Activin A-specific binding proteins”. Activins are homo- and hetero-dimeric molecules comprising beta subunits, i.e., InhibinβA, inhibinβB, inhibinβC, and/or inhibinβE. The βA subunit has the amino acid sequence of SEQ ID NO:226 and the βB subunit has the amino acid sequence of SEQ ID NO:228. Activin A is a homodimer of two βA subunits; Activin B is a homodimer of two βB subunits; Activin AB is a heterodimer of one βA subunit and one βB subunit; and Activin AC is a heterodimer of one βA subunit and one βC subunit. An Activin A-specific binding protein may be an antigen-specific binding protein that specifically binds the βA subunit. Since the βA subunit is found in Activin A, Activin AB, and Activin AC molecules, an “Activin A-specific binding protein” can be an antigen-specific binding protein that specifically binds Activin A as well as Activin AB and Activin AC (by virtue of its interaction with the βA subunit).

In one embodiment of the present invention, an Activin A-specific binding protein specifically binds Activin A; or Activin A and Activin AB; or Activin A and Activin AC; or Activin A, Activin AB and Activin AC, but does not bind other ActRIIB ligands such as Activin B, GDF3, GDF8, BMP2, BMP4, BMP7, BMP9, BMP10, GDF11, Nodal, etc. In another embodiment, an Activin A-specific binding protein specifically binds to Activin A but does not bind significantly to Activin B or Activin C. In another embodiment, an Activin A-specific binding protein may also bind to Activin B (by virtue of cross-reaction with the εB subunit, i.e., InhibinβB). In another embodiment, an Activin A-specific binding protein is a binding protein that binds specifically to Activin A but does not bind to any other ligand of ActRIIB. In another embodiment, an Activin A-specific binding protein is a binding protein and binds specifically to Activin A and does not bind to any Bone Morphogenetic Protein (BMP) (e.g., BMP2, BMP4, BMP6, BMP9, BMP10). In another embodiment, an Activin A-specific binding protein is a binding protein that binds specifically to Activin A but does not bind to any other member of the transforming growth factor beta (TGFβ) superfamily.

The term “specifically binds” or the like, as used herein, means that an antigen-specific binding protein, or an antigen-specific binding domain, forms a complex with a particular antigen characterized by a dissociation constant (K_{D}) of 500 pM or less, and does not bind other unrelated antigens under ordinary test conditions. “Unrelated antigens” are proteins, peptides or polypeptides that have less than 95% amino acid identity to one another. Methods for determining whether two molecules specifically bind one another are well known in the art and include, for example, equilibrium dialysis, surface plasmon resonance, and the like. For example, an antigen-specific binding protein or an antigen-specific binding domain, as used in the context of the present invention, includes molecules that bind a particular antigen (e.g., Activin A and/or AB, or GDF8) or a portion thereof with a K_{D} of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

As used herein, an antigen-specific binding protein or antigen-specific binding domain “does not bind” to a specified molecule (e.g., “does not bind GDF11,” “does not bind BMP9,” “does not bind BMP10,” etc.) if the protein or binding domain, when tested for binding to the molecule at 25° C. in a surface plasmon resonance assay, exhibits a K_{D} of greater than 50.0 nM, or fails to exhibit any binding in such an assay or equivalent thereof.

The term “surface plasmon resonance,” as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D},” as used herein, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

In one embodiment, an antigen-specific binding protein for use in the methods of the present invention may comprise or consist of an antibody or antigen-binding fragment of an antibody.

As used herein, the term “an antibody that binds Activin” or an “anti-Activin A antibody” includes antibodies, and antigen-binding fragments thereof, that bind a soluble fragment of the Activin A protein and may also bind to an ActivinβA subunit-containing Activin heterodimer.

The term “antibody” as used herein, means any antigen-binding molecule or molecular complex comprising at least one complementarity determining region (CDR) that specifically binds to or interacts with a particular antigen (e.g., Activin A). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-Activin A antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL.

In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The antibodies of the present invention may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al., PNAS USA 95:652-656 (1998)). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments of the invention, the anti-Activin A antibodies are human antibodies. The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., Nucl Acids Res 20:6287-6295 (1992)) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. Molecular Immunology 30:105 1993)) to levels typically observed using a human IgG1 hinge. The instant invention encompasses antibodies having one or more mutations in the hinge, CH2 or CH3 region which may be desirable, for example, in production, to improve the yield of the desired antibody form.

The antibodies for use in the methods of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present invention includes neutralizing and/or blocking anti-Activin A antibodies. A “neutralizing” or “blocking” antibody, as used herein, is intended to refer to an antibody whose binding to Activin A: (i) interferes with the interaction between Activin A and an Activin A receptor (e.g., Activin Type IIA receptor, Activin Type IIB receptor, Activin Type I receptor, etc.); (ii) interferes with the formation of Activin-Activin receptor complexes; and/or (iii) results in inhibition of at least one biological function of Activin A. The inhibition caused by an Activin A neutralizing or blocking antibody need not be complete so long as it is detectable using an appropriate assay.

The anti-Activin A antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-Activin A antibodies comprising variants of any of the HCVR, LCVR, and/or CDR amino acid sequences disclosed herein having one or more conservative substitutions. For example, the present invention includes anti-Activin A antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, W.R., Methods Mol Biol 24: 307-331 (1994), herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al., Science 256: 1443-1445 (1992), herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (see, e.g., Pearson, W.R., Methods Mol Biol 132: 185-219 (2000), herein incorporated by reference). Another preferred algorithm when comparing a sequence of the invention to a database containing a large number of sequences from different organisms is the computer program BLAST, especially BLASTP or TBLASTN, using default parameters. See, e.g., Altschul et al., J Mol Biol 215:403-410 (1990) and Altschul et al., Nucleic Acids Res 25:3389-402 (1997), each herein incorporated by reference.

Suitable anti-Activin A antibodies, and antigen-binding fragments thereof, that bind Activin A with high affinity are also suitable for use in the methods of the present invention. For example, the present invention includes antibodies and antigen-binding fragments of antibodies that bind Activin A (e.g., at 25° C. or 37° C.) with a K_{D} of less than about 30 nM as measured by surface plasmon resonance. In certain embodiments, the antibodies or antigen-binding fragments of the present invention bind Activin A with a K_{D} of less than about 25 nM, less than about 20 nM, less than about 15 nM, less than about 10 nM, less than about 5 nM, less than about 2 nM, less than about 1 nM, less than about 500 pM, less than about 250 pM, less than about 240 pM, less than about 230 pM, less than about 220 pM, less than about 210 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 95 pM, less than about 90 pM, less than about 85 pM, less than about 80 pM, less than about 75 pM, less than about 70 pM, less than about 65 pM, less than about 60 pM, less than about 55 pM, less than about 50 pM, less than about 45 pM, less than about 40 pM, less than about 35 pM, less than about 30 pM, less than about 25 pM, less than about 20 pM, less than about 15 pM, less than about 10 pM, less than about 9 pM, less than about 8 pM, less than about 7 pM, less than about 6 pM, less than about 5 pM, less than about 4 pM, or less than about 3 pM, as measured by surface plasmon resonance.

The present invention also includes anti-Activin A antibodies, and antigen-binding fragments thereof, that inhibit Activin A-mediated cellular signaling. For example, the present invention includes anti-Activin A antibodies that inhibit the activation of the SMAD complex signal transduction pathway via the binding of Activin A to Activin Type I or II receptors with an IC₅₀ value of less than about 4 nM, as measured in a cell-based blocking bioassay. In certain embodiments, the antibodies or antigen-binding fragments of the present invention inhibit the activation of the SMAD complex signal transduction pathway via the binding of Activin A to Activin Type I or II receptors with an IC₅₀ value of less than about 3 nM, less than about 2 nM, less than about 1 nm, less than about 500 pM, less than about 250 pM, less than about 240 pM, less than about 230 pM, less than about 220 pM, less than about 210 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 95 pM, less than about 90 pM, less than about 85 pM, less than about 80 pM, less than about 75 pM, less than 70 pM, less than about 65 pM, less than about 60 pM, less than about 55 pM, less than about 50 pM, less than about 49 pM, less than about 48 pM, less than about 47 pM, less than about 46 pM, less than about 45 pM, less than about 44 pM, less than about 43 pM, less than about 42 pM, less than about 41 pM, less than about 40 pM, or less than about 39 pM, as measured in a cell-based blocking bioassay.

In certain embodiments, the antibodies or antigen-binding fragments of the present invention inhibit the signaling activing of Activin B by interfering with the binding of Activin B to Activin Type I or II receptors with an IC₅₀ value of less than about 50 nM, less than about 20 nM, less than about 10 nm, less than about 5 nM, or less than about 1 nM, as measured in a cell-based blocking bioassay. In certain embodiments, the antibodies or antigen-binding fragments of the present invention inhibit the activation of the SMAD complex signal transduction pathway via the binding of Activin AB to Activin Type I or II receptors with an IC₅₀ value of less than about 500 pM, less than about 450 pM, less than about 440 pM, less than about 430 pM, less than about 420 pM, less than about 410 pM, less than about 400 pM, less than about 390 pM, less than about 380 pM, less than about 370 pM, less than about 360 pM, less than about 350 pM, less than about 340 pM, less than about 320 pM, less than about 310 pM, less than about 300 pM, less than about 290 pM, less than about 280 pM, less than about 270 pM, less than about 260 pM, less than about 250 pM, less than about 240 pM, less than about 230 pM, less than about 220 pM, less than about 210 pM, less than about 200 pM, less than about 190 pM, less than about 180 pM, less than about 170 pM, less than about 160 pM, less than about 150 pM, or less than about 140 pM, as measured in a cell-based blocking bioassay. In certain embodiments, the antibodies or antigen-binding fragments of the present invention inhibit the activation of the SMAD complex signal transduction pathway via the binding of Activin AC to Activin Type I or II receptors with an IC50 value of less than about 1 nM, less than about 900 pM, less than about 800 pM, less than about 750 pM, less than about 700 pM, less than about 650 pM, less than about 600 pM, or less than about 580 pM, as measured in a cell-based blocking bioassay.

The antibodies, or antigen-binding fragments thereof, for use in the present invention may possess one or more of the aforementioned biological characteristics, or any combinations thereof. Other biological characteristics of the antibodies will be evident to a person of ordinary skill in the art from a review of the present disclosure including the working Examples herein.

In some embodiments, anti-Activin A antibodies for use in the present invention comprise an Fc domain comprising one or more mutations which enhance or diminish antibody binding to the FcRn receptor, e.g., at acidic pH as compared to neutral pH. For example, the present invention includes anti-Activin A antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-Activin A antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). All possible combinations of the foregoing Fc domain mutations, and other mutations within the antibody variable domains disclosed herein, are contemplated within the scope of the present invention.

The present invention also includes anti-Activin A antibodies comprising a chimeric heavy chain constant (CH) region, wherein the chimeric CH region comprises segments derived from the CH regions of more than one immunoglobulin isotype. For example, antibodies for use in the invention may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human

IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., U.S. Provisional Appl. No. 61/759,578, filed Feb. 1, 2013, the disclosure of which is hereby incorporated by reference in its entirety).

Anti-Activin A antibodies which interact with one or more amino acids found within Activin A (e.g., within the Activin Type II receptor binding site) are also suitable for use in the present invention. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within the Activin βA subunit. Alternatively, the epitope may consist of a plurality of non-contiguous amino acids (or amino acid sequences) located within the Activin A dimer.

Various techniques known to persons of ordinary skill in the art can be used to determine whether an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., N.Y.), alanine scanning mutational analysis, peptide blots analysis (Reineke, Methods Mol Biol 248:443-463 (2004)), and peptide cleavage analysis. In addition, methods such as epitope excision, epitope extraction and chemical modification of antigens can be employed (Tomer, Protein Science 9:487-496 (2000)). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring, Analytical Biochemistry 267(2):252-259 (1999); Engen and Smith, Anal. Chem. 73:256A-265A (2001).

Anti-Activin A antibodies that bind to the same epitope as any of the specific exemplary antibodies described herein (e.g., H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P2, H4H10447P2, H4H10448P2, H4H10452P2, H4H10468P2, H2aM10965N, etc.) are envisioned for use in the methods of the invention. Likewise, the present invention also includes anti-Activin A antibodies that compete for binding to Activin A with any of the specific exemplary antibodies described herein (e.g., H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P2, H4H10447P2, H4H10448P2, H4H10452P2, H4H10468P2, H2aM10965N, etc.). For example, the present invention includes use of anti-Activin A antibodies that cross-compete for binding to Activin A with one or more antibodies selected from the group consisting of H4H10423P, H4H10446P2, H4H10468P2 and H4H10442P2. The present invention also includes anti-Activin A antibodies that cross-compete for binding to Activin A with one or more antibodies selected from the group consisting of H4H10429, H4H1430P, H4H10432P2, H4H10436P2, and H4H10440P2.

One can easily determine whether an antibody binds to the same epitope as, or competes for binding with, a reference anti-Activin A antibody by using routine methods known in the art. For example, to determine if a test antibody binds to the same epitope as a reference anti-Activin A antibody of the invention, the reference antibody is allowed to bind to Activin A (or a βA subunit-containing heterodimer). Next, the ability of a test antibody to bind to Activin A is assessed. If the test antibody is able to bind to Activin A following saturation binding with the reference anti-Activin A antibody, it can be concluded that the test antibody binds to a different epitope than the reference anti-Activin A antibody. On the other hand, if the test antibody is not able to bind to Activin A following saturation binding with the reference anti-Activin A antibody, then the test antibody may bind to the same epitope as the epitope bound by the reference anti-Activin A antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 50:1495-1502 (1990)). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-Activin A antibody, the above-described binding methodology is performed in two orientations: In a first orientation, the reference antibody is allowed to bind to Activin A protein (or a βA subunit-containing heterodimer) under saturating conditions followed by assessment of binding of the test antibody to the Activin A molecule. In a second orientation, the test antibody is allowed to bind to Activin A under saturating conditions followed by assessment of binding of the reference antibody to Activin A. If, in both orientations, only the first (saturating) antibody is capable of binding to Activin A, then it is concluded that the test antibody and the reference antibody compete for binding to Activin A. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

Anti-Activin A antibodies of the invention may bind to an epitope on Activin A that is within or near the binding site for an Activin Type II receptor, directly block interaction between Activin A and an Activin Type II receptor, and indirectly block interaction between Activin A and an Activin Type I receptor. Anti-Activin A antibodies of the invention may bind to an epitope on Activin A that is within or near the binding site for the Activin Type I receptor and directly block interaction between Activin A and an Activin Type I receptor. In one embodiment of the invention, an anti-Activin A antibody that binds to Activin A at or near the Activin Type I receptor binding site does not block interaction between Activin A and an Activin A Type II receptor.

The anti-Activin A antibodies and antibody fragments of the present invention encompass proteins having amino acid sequences that vary from those of the described antibodies but that retain the ability to bind human Activin A. Such variant antibodies and antibody fragments comprise one or more additions, deletions, or substitutions of amino acids when compared to parent sequence, but exhibit biological activity that is essentially equivalent to that of the described antibodies. Likewise, the anti-Activin A antibody-encoding DNA sequences of the present invention encompass sequences that comprise one or more additions, deletions, or substitutions of nucleotides when compared to the disclosed sequence, but that encode an anti-Activin A antibody or antibody fragment that is essentially bioequivalent to an anti-Activin A antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-Activin A antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-Activin A antibody variants comprising amino acid changes which modify the glycosylation characteristics of the antibodies, e.g., mutations which eliminate or remove glycosylation.

An anti-Activin A antibody, or antigen-binding fragment thereof, for use in the methods of the present invention may be present in a pharmaceutical composition. Such pharmaceutical compositions are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA, J Pharm Sci Technol 52:238-311 (1998).

Various delivery systems are known and can be used to administer a pharmaceutical composition comprising an anti-Activin A antibody, or antigen-binding fragment thereof, e.g., encapsulation in liposomes, microparticles, microcapsules, recombinant cells capable of expressing the antibody, receptor mediated endocytosis (see, e.g., Wu et al., J Biol Chem 262:4429-4432 (1987)). The antibodies may also be delivered by gene therapy techniques. Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition comprising an anti-Activin A antibody, or antigen-binding fragment thereof, can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, CRC Crit. Ref Biomed. Eng. 14:201 (1987)). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, Science 249:1527-1533 (1990).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

Methods for generating monoclonal antibodies, including fully human monoclonal anti-Activin A antibodies, or antigen-binding fragments thereof, suitable for use in the methods of the present invention are known in the art. Any such known methods can be used in the context of the present invention to make human antibodies that specifically bind to human Activin A.

Using VELOCIMMUNE™ technology, for example, or any other known method for generating fully human monoclonal antibodies, high affinity chimeric antibodies to human Activin A are initially isolated having a human variable region and a mouse constant region. As in the experimental section below, the antibodies are characterized and selected for desirable characteristics, including affinity, selectivity, epitope, etc. If necessary, mouse constant regions are replaced with a desired human constant region, for example wild-type or modified IgG1 or IgG4, to generate a fully human anti-Activin A antibody. While the constant region selected may vary according to specific use, high affinity antigen-binding and target specificity characteristics reside in the variable region. In certain instances, fully human anti-Activin A antibodies are isolated directly from antigen-positive B cells.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Example 1

### Activin A Binding Proteins

U.S. Patent Publication No. 2015/0037339, the entire contents of which are incorporated herein by reference, describes the generation and characterization of fully human anti-Activin A antibodies (i.e., antibodies possessing human variable domains and human constant domains) suitable for use in the present invention. Exemplary antibodies include those designated as: H4H10423P, H4H10429P, H4H10430P, H4H10432P2, H4H10440P2, H4H10442P2, H4H10436P2, and H4H10446P2.

Table 1 provides the heavy and light chain variable region amino acid sequence pairs of selected anti-Activin A antibodies and their corresponding antibody identifiers. The corresponding nucleic acid sequence identifiers are set forth in Table 2.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H10423P | 2 | 4 | 6 | 8 | 10 | 12 | 14 | 16 |
| H4H10424P | 18 | 20 | 22 | 24 | 26 | 28 | 30 | 32 |
| H4H10426P | 34 | 36 | 38 | 40 | 42 | 44 | 46 | 48 |
| H4H10429P | 50 | 52 | 54 | 56 | 58 | 60 | 62 | 64 |
| H4H10430P | 66 | 68 | 70 | 72 | 74 | 76 | 78 | 80 |
| H4H10432P2 | 82 | 84 | 86 | 88 | 90 | 92 | 94 | 96 |
| H4H10433P2 | 98 | 100 | 102 | 104 | 90 | 92 | 94 | 96 |
| H4H10436P2 | 106 | 108 | 110 | 112 | 90 | 92 | 94 | 96 |
| H4H10437P2 | 114 | 116 | 118 | 120 | 90 | 92 | 94 | 96 |
| H4H10438P2 | 122 | 124 | 126 | 128 | 90 | 92 | 94 | 96 |
| H4H10440P2 | 130 | 132 | 134 | 136 | 90 | 92 | 94 | 96 |
| H4H10442P2 | 138 | 140 | 142 | 144 | 146 | 148 | 150 | 152 |
| H4H10445P2 | 154 | 156 | 158 | 160 | 146 | 148 | 150 | 152 |
| H4H10446P2 | 162 | 164 | 166 | 168 | 146 | 148 | 150 | 152 |
| H4H10447P2 | 170 | 172 | 174 | 176 | 146 | 148 | 150 | 152 |
| H4H10448P2 | 178 | 180 | 182 | 184 | 146 | 148 | 150 | 152 |
| H4H10452P2 | 186 | 188 | 190 | 192 | 146 | 148 | 150 | 152 |
| H4H10468P2 | 194 | 196 | 198 | 200 | 146 | 148 | 150 | 152 |
| H2aM10965N | 202 | 204 | 206 | 208 | 210 | 212 | 214 | 216 |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H10423P | 1 | 3 | 5 | 7 | 9 | 11 | 13 | 15 |
| H4H10424P | 17 | 19 | 21 | 23 | 25 | 27 | 29 | 31 |
| H4H10426P | 33 | 35 | 37 | 39 | 41 | 43 | 45 | 47 |
| H4H10429P | 49 | 51 | 53 | 55 | 57 | 59 | 61 | 63 |
| H4H10430P | 65 | 67 | 69 | 71 | 73 | 75 | 77 | 79 |
| H4H10432P2 | 81 | 83 | 85 | 87 | 89 | 91 | 93 | 95 |
| H4H10433P2 | 97 | 99 | 101 | 103 | 89 | 91 | 93 | 95 |
| H4H10436P2 | 105 | 107 | 109 | 111 | 89 | 91 | 93 | 95 |
| H4H10437P2 | 113 | 115 | 117 | 119 | 89 | 91 | 93 | 95 |
| H4H10438P2 | 121 | 123 | 125 | 127 | 89 | 91 | 93 | 95 |
| H4H10440P2 | 129 | 131 | 133 | 135 | 89 | 91 | 93 | 95 |
| H4H10442P2 | 137 | 139 | 141 | 143 | 145 | 147 | 149 | 151 |
| H4H10445P2 | 153 | 155 | 157 | 159 | 145 | 147 | 149 | 151 |
| H4H10446P2 | 161 | 163 | 165 | 167 | 145 | 147 | 149 | 151 |
| H4H10447P2 | 169 | 171 | 173 | 175 | 145 | 147 | 149 | 151 |
| H4H10448P2 | 177 | 179 | 181 | 183 | 145 | 147 | 149 | 151 |
| H4H10452P2 | 185 | 187 | 189 | 191 | 145 | 147 | 149 | 151 |
| H4H10468P2 | 193 | 195 | 197 | 199 | 145 | 147 | 149 | 151 |
| H2aM10965N | 201 | 203 | 205 | 207 | 209 | 211 | 213 | 215 |

Antibodies are typically referred to herein according to the following nomenclature: Fc prefix (e.g. “H1M,” “H2aM,” “H4H”), followed by a numerical identifier (e.g. “10423,” “10424,” or “10426” as shown in Tables 1 and 2), followed by a “P,” “P2” or “N” suffix. Thus, according to this nomenclature, an antibody may be referred to herein as, e.g.,”H4H10423P,” “H4H10432P2,” “H2aM10965N,” etc. The H1M, H2M and H4H prefixes on the antibody designations used herein indicate the particular Fc region isotype of the antibody. For example, an “H2aM” antibody has a mouse IgG2a Fc, whereas an “H4H” antibody has a human IgG4 Fc. As will be appreciated by a person of ordinary skill in the art, an antibody having a particular Fc isotype can be converted to an antibody with a different Fc isotype (e.g., an antibody with a mouse IgG2a Fc can be converted to an antibody with a human IgG4, etc.), but in any event, the variable domains (including the CDRs)—which are indicated by the numerical identifiers shown in Table 1 —will remain the same, and the binding properties are expected to be identical or substantially similar regardless of the nature of the Fc domain.

### Example 2

### Anti-Activin A Antibody Treatment Preserves Pulmonary Artery Size and Right Ventricular Stroke Volume in a Mouse Model of Chronic Hypoxia

To evaluate the effect of anti-Activin A antibodies in pulmonary arterial hypertension, a 4 week chronic hypoxia-induced pulmonary arterial hypertension mouse model was used.

The following materials and methods were used for this study.

Materials and Methods

Mice

Eleven to fourteen week old Taconic C57BL/6 mice were used for the study. Mice were separated into treatment groups by weight such that starting body weights were similar among different groups. Cages were selected to either remain at ˜21% O₂ (normobaric normoxia) or placed into 10% O₂ (normobaric hypoxia) chamber (a modified 3′ Semi-Rigid Isolator unit, Charles River) that maintained low O₂ levels with adjustment of N₂ flow to a steady intake of room air.

As outlined in Table 3, mice were subcutaneously administered 25 mg/kg of REGN2477, (H4H10446P2), 25 mg/kg of an isotype control antibody, or 10 μL/kg of saline starting on day 14.

TABLE 3
Therapeutic dosing and treatment protocol for each
group in chronic hypoxia mouse model studies
Study 1: 4 week chronic hypoxia with
drug dosing beginning after 14 days in hypoxia

| | | | | | | Number |
|---|---|---|---|---|---|---|
| | | | | | | of mice/ |
| | | | | | | group |
| Group | Condition | Treatment | Dosage | Frequency | Route | (“n” size) |
| 1 | Normobaric | Saline | 5 mL/kg | 2x/week | SC | 10 |
| | normoxia | | | | | |
| 2 | Normobaric | Saline | 5 mL/kg | 2x/week | SC | 10 |
| | hypoxia | | | | | |
| 3 | Normobaric | Isotype | 25 mg/kg | 2x/week | SC | 10 |
| | hypoxia | control | | | | |
| | | antibody | | | | |
| 4 | Normobaric | Anti- | 25 mg/kg | 2x/week | SC | 10 |
| | hypoxia | Activin A | | | | |
| | | antibody | | | | |

SC = subcutaneous

Ultrasound Assessment and Analysis

On the last day of each study, pulmonary artery size and right ventricular function and dimensions were assessed in each mouse using a high frequency ultrasound system (Vevo 2100, VisualSonics). For the assessment, mice were anesthetized (with 1.5% isoflurane at a rate of 1.0 cc/mL of medical grade air) and their temperature was monitored with a rectal temperature probe and held at approximately 37° C. with a heated platform (MouseMonitorS, Indus Instruments) and a warming lamp. Both brightness-mode (B-mode) and motion-mode (M-mode) imaging were used. B-mode imaging of the mouse heart in cross-section was used to determine pulmonary artery cross-sectional area (PA CSA) at the level of the pulmonary valve. M-mode imaging was used to determine the pulsed wave velocity time integral (VTI), which is derived from the area under the curve of representative Doppler tracings of blood flow through the pulmonary artery. Right ventricular stroke volume (RV SV) was calculated from the product of PA CSA and VTI. Right ventricular cardiac output (RV CO) was calculated from the product of SV and heart rate (HR). M-mode imaging was used to determine right ventricular free wall (RVFW) thickness during diastole and systole. Animals were returned to their home cages before right ventricular pressure assessment.

Right Ventricular Pressure Assessment

Right ventricular pressure was subsequently assessed for all treatment groups. Mice were anesthetized with isoflurane and were kept at approximately 37° C. using a heated platform (Heated Hard Pad 1, Braintree Scientific) and circulating heated water pump (T/Pump Classic, Gaymar Industries). The neck area for each mouse was prepared for surgery by depilating over the right common Carotid artery and right Jugular vein. An incision was made and the right Jugular vein was isolated with care as to not damage the Carotid artery and/or the Vagus nerve. A piece of 5-0 silk suture was placed under the isolated Jugular vein to allow for retraction of the vessel cranially, then a 30-guage needle was used to introduce a hole into the Jugular vein. A pressure catheter (Micro-tip catheter transducer SPR-1000, Millar Instruments, Inc.) was inserted into the opening of the Jugular vein and advanced past the right atrium into the right ventricle. The catheter was connected to pressure/volume instrument (MPVS-300, Millar Instruments, Inc.) that measured heart rate as well as both diastolic and systolic right ventricular pressures. These parameters were digitally acquired using a data acquisition system (PowerLab 4/35, ADlnstruments). LabChart Pro 7.0 software (ADlnstruments) was used to analyze right ventricular pressures. Readings were quantified from a 60 second interval of the pressure tracing (following a 2 minute period of recording to allow for pressure stabilization). The parameters analyzed were right ventricular systolic pressures (RVSP), heart rate (HR) and rate of right ventricular pressure rise (dP/dt max).

Serum/Tissue Collection and Assessment of Right Ventricular Hypertrophy

Following completion of right ventricular pressure measurement, the catheter was removed and each animal was sacrificed. The abdomen was opened and blood was drawn from the Vena Cava for hematocrit assessment and serum collection. The thoracic cavity was then opened and the middle lobe of the right lung was ligated with 5-0 silk suture, excised, placed in RNA later (Sigma-Aldrich, cat #R0901) and frozen at −80° C. 24 hours later. The heart was excised from each animal, and the right ventricle (RV) was carefully cut away from the left ventricle and septum (LV+S). Both pieces of heart tissue were separately weighed on a micro-balance (AJ000, Mettler) to calculate the index of RV hypertrophy [RV/(LV+S); Fulton Index].

Half of the animals from each treatment group had the lungs perfused at 20-25 mmHg with phosphate buffered solution (PBS, pH 7.4), then fixed with 10% neutral-buffered formalin (NBF). Lungs remained in 10% NBF for 24 hours before being placed into 70% ethanol for at least 48 hours, before tissue processing and paraffin embedding. For animals that did not undergo perfusion-fixation of the lung, the right inferior lobe was ligated with 5-0 silk suture before being excised, weighed and frozen in liquid N2.

### Results

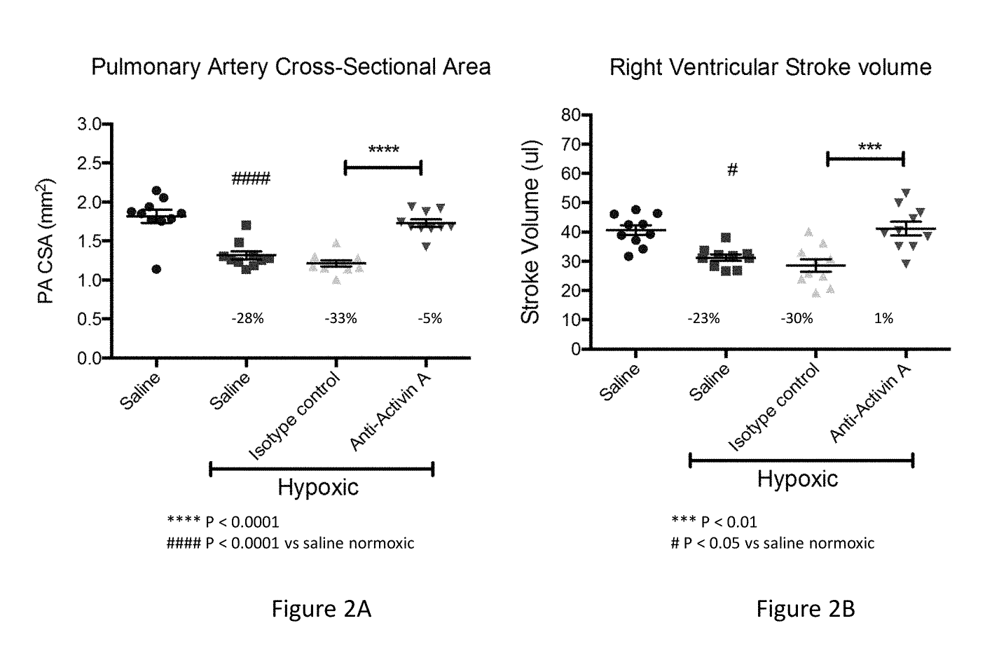

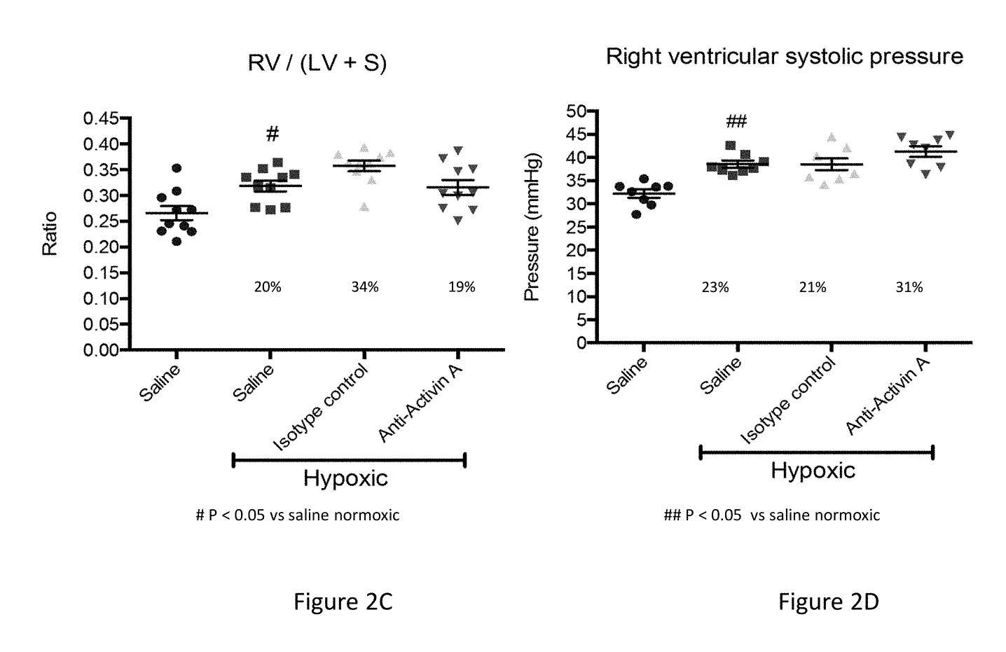

As shown in FIG. 2A, B-mode ultrasound imaging of the mouse heart in cross-section revealed that a 4 week exposure to hypoxia reduced PA cross-sectional area (CSA) in saline-treated mice by ˜28% relative to normoxic mice. Under hypoxic conditions, treatment with the isotype control antibody showed a similar response as the saline-treated group. Therapeutic treatment with the anti-Activin A antibody in hypoxia restored PA CSA to values measured in normoxic saline-treated mice (1.727±0.048 mm2 and 1.817±0.085 mm2, respectively).

Ultrasound M-mode imaging of the pulsed wave VTI found non-significant reductions in the blood flow velocity through the pulmonary artery in animals exposed to chronic hypoxia (data not shown). Calculated right ventricular stroke volume in mice exposed to hypoxia was significantly reduced by 23-29% (both saline- and isotype control-treated mice); however, mice treated with anti-Activin A antibody had stroke volumes comparable to volumes measured for the normoxic saline-treated group, as shown in Table 4 and FIG. 2B. The measured right ventricular stroke volume in the anti-Activin A-treated group was significantly greater than the stroke volume observed for the isotype control group (41.19±2.34 ul vs 28.55±2.14 ul, respectively). Heart rate was measured and not found to be significantly different among groups. Right ventricular cardiac output was ˜21% lower in animals exposed to chronic hypoxia relative to animals in normoxia. Use of anti-Activin A antibody in hypoxia resulted in cardiac outputs that were significantly greater than those measured for the isotype control antibody group (20.70±1.41 ml/min vs 13.53±0.84 ml/min, respectively).

TABLE 4
Stroke Volumes

| | | | | | | Right |
|---|---|---|---|---|---|---|
| | | | | Stroke | | Ventricular |
| | | | PA CSA | Volume | Heart rate | cardiac output |
| | | | (mm²) | (uL) | (beats/min) | (mL/min) |
| Group | Condition | Treatment | (Mean ± SEM) | (Mean ± SEM) | (Mean ± SEM) | (Mean ± SEM) |
| 1 | Normobaric | Saline | 1.817 ± 0.085 | 40.64 ± 1.69 | 464.2 ± 9.5 | 18.84 ± 0.83 |
| | normoxia | | | | | |
| 2 | Normobaric | Saline | 1.315 ± 0.052**** | 31.26 ± 1.09** | 475.1 ± 16.7 | 14.89 ± 0.82* |
| | hypoxia | | | | | |
| 3 | Normobaric | Isotype | 1.213 ± 0.039 | 28.55 ± 2.14 | 481.7 ± 20.5 | 13.53 ± 0.84 |
| | hypoxia | control | | | | |
| | | antibody | | | | |
| 4 | Normobaric | Anti- | 1.727 ± 0.048^{####} | 41.19 ± 2.34^{####} | 500.9 ± 15.6 | 20.70 ± 1.41^{####} |
| | hypoxia | Activin A | | | | |
| | | antibody | | | | |

One-way ANOVA with Sidak's multiple comparison test: *, **, ***, **** for P < 0.05, 0.01, 0.001, 0.0001 vs. normobaric normoxia saline-treated; #, ##, #### for P < 0.05, 0.01, 0.001 vs. normobaric hypoxia isotype control antibody-treated; %, %% for P < 0.05, 0.01 vs. normobaric hypoxia saline treated.

Ultrasound M-mode imaging of the right ventricular wall revealed that measured wall thicknesses during systole and diastole were not statistically reduced when anti-Activin A antibodies were used in hypoxia (FIG. 2C).

Catheter-based assessment of heart rate and right ventricular pressures revealed a significant elevation of heart rate and significantly higher systolic pressures in the hypoxic saline-treated than normoxic animals (FIG. 2D). Similarly, the isotype antibody-treated group in hypoxia showed elevated heart rates and systolic pressures, but anti-Activin A treatment did not reduce right ventricular systolic pressure elevation (FIG. 2D).

Under hypoxic conditions, erythropoietin is synthesized and released by the kidney to induce the production of red blood cells (RBCs) for increased oxygen delivery. Animals exposed to chronic hypoxia showed an increase in RBCs as assessed by hematocrit. Treatment with anti-Activin A further increased the hematocrit compared to the isotype control antibody treatment as shown in Table 5.

TABLE 5
Average hemocrit levels of treatment groups at end of each study

| | | | Hemocrit |
|---|---|---|---|
| Group | Condition | Treatment | (Mean ± SEM) |
| 1 | Normobaric normoxia | Saline | 41.6 ± 0.7 |
| 2 | Normobaric hypoxia | Saline | 51.3 ± 0.4**** |
| 3 | Normobaric hypoxia | Isotype control | 50.5 ± 1.0 |
| | | antibody | |
| 4 | Normobaric hypoxia | Anti-Activin A | 54.6 ± 0.8^{####} |
| | | antibody | |

One-way ANOVA with Sidak's multiple comparison test:
****for P < 0.0001 vs. normobaric normoxia saline-treated;
^{####}for P < 0.0001 vs. normobaric hypoxia isotype control antibody-treated.

### Example 3

### Anti-Activin A Antibody Treatment Restores Pulmonary Artery Size and Right Ventricular Cardiac Function in a Rat Model of Pulmonary Hypertension Induced by Monocrotaline

To further evaluate the effect of anti-Activin A antibodies in pulmonary arterial hypertension, a rat model of pulmonary hypertension induced by monocrotaline was used.

The following materials and methods were used for this study.

### Materials and Methods

Rats

Six to seven week old Sprague Dawley rats were used. Rats were separated into treatment groups such that body weights were similar among different groups. Rats were subcutaneously administered either 40 mg/kg of monocrotaline or 5 mL/kg of saline at day 0. At 14 days post injection, saline-injected rats were orally dosed with PEG 400 (at 50:50 v/v, Affymetrix Inc., #19957) at 5 mL/kg daily for two weeks, and monocrotaline-injected rats were separated into 5 groups with a group (n=9) orally dosed with PEG 400 at 5 mL/kg daily for two weeks, another group (n=12) orally administered macintentan at 30 mg/kg daily for two weeks, and three groups that were both orally dosed with PEG 400 at 5 mL/kg daily for two weeks and subcutaneously treated with either anti-Activin A antibodies, H4H10430P (n=9), H4H10446P2 (n=11), or an isotype control antibody (n=10) at 40 mg/kg twice a week for two weeks. Experimental dosing and treatment protocol for groups of rats are shown in Table 6.

TABLE 6
Therapeutic dosing and treatment protocol for
each group in rat monocrotaline model

| | | | | | | “n” |
|---|---|---|---|---|---|---|
| Group | Condition | Treatment | Dosage | Frequency | Route | size |
| 1 | Saline | PEG 400 (50:50) | 5 mL/kg | Daily | PO | 9 |
| 2 | Monocrotaline | PEG 400 (50:50) | 5 mL/kg | Daily | PO | 12 |
| 3 | Monocrotaline | Macitentan | 30 mg/kg | Daily | PO | 12 |
| 4 | Monocrotaline | PEG 400 (50:50) | 5 mL/kg | Daily | PO | 10 |
| | | Isotype control antibody | 40 mg/kg | 2x/wk | SC | |
| 5 | Monocrotaline | PEG 400 (50:50) | 5 mL/kg | Daily | PO | 9 |
| | | H4H10430P | 40 mg/kg | 2x/wk | SC | |
| 6 | Monocrotaline | PEG 400 (50:50) | 5 mL/kg | Daily | PO | 11 |
| | | H4H10446P2 | 40 mg/kg | 2x/wk | SC | |

Ultrasound Assessment and Analysis

At day 27, pulmonary artery size and right ventricular function and dimensions of the rats were assessed using a high frequency ultrasound system (Vevo 2100, VisualSonics). For the assessment, rats were anesthetized (with 1.5% isoflurane at a rate of 1.0 cc/mL of medical grade air) and their temperature was monitored with a rectal temperature probe and held at approximately 37° C. with a heated platform (Vevo 2100, Visualsonics) and a warming lamp. Both brightness-mode (B-mode) and motion-mode (M-mode) imaging were used. B-mode imaging of the rat heart in cross-section was used to determine pulmonary artery cross-sectional area (PA CSA) at the level of the pulmonary valve. M-mode imaging was used to determine the pulsed wave velocity time integral (VTI), which is derived from the area under the curve of representative Doppler tracings of blood flow through the pulmonary artery. Right ventricular stroke volume (RV SV) was calculated from the product of PA CSA and VTI. Right ventricular cardiac output (RV CO) was calculated from the product of SV and heart rate (HR). M-mode imaging was used to determine right ventricular free wall (RVFW) thickness during diastole and systole. Animals were returned to their home cages before right ventricular pressure assessment.

Right Ventricular Pressure Assessment

Right ventricular pressure was subsequently assessed for all treatment groups. Rats were anesthetized with isoflurane and were kept at approximately 37° C. using a platform (Heated Hard Pad 1, Braintree Scientific) and circulating heated water pump (T/Pump Classic, Gaymar Industries). The neck area for each rat was prepared for surgery by depilating over the right common carotid artery and right jugular vein. An incision was made and the right jugular vein was isolated with care as to not damage the carotid artery and/or the Vagus nerve. A piece of 5-0 silk suture was placed under the isolated jugular vein to allow for retraction of the vessel cranially, then a 26-guage needle was used to introduce a hole into the jugular vein. A pressure catheter (Micro-tip catheter transducer SPR-1000, Millar Instruments, Inc.) was inserted into the opening of the jugular vein and advanced past the right atrium into the right ventricle. The catheter was connected to pressure/volume instrument (MPVS-300, Millar Instruments, Inc.) that measured heart rate as well as both diastolic and systolic right ventricular pressures. These parameters were digitally acquired using a data acquisition system (PowerLab 4/35, ADlnstruments). LabChart Pro 7.0 software (ADlnstruments) was used to analyze right ventricular pressures. Readings were quantified from a 60 second interval of the pressure tracing (following a 2 minute period of recording to allow for pressure stabilization). The parameters analyzed were RV diastolic and systolic pressures, heart rate and ventricular contractile function.

Serum/Tissue Collection and Assessment of Right Ventricular Hypertrophy

Following completion of right ventricular pressure measurement, the catheter was removed and each animal was sacrificed. The abdomen was opened and blood was drawn from the vena cava for hematocrit assessment and serum collection. The thoracic cavity was then opened and the middle lobe of the right lung was ligated with 5-0 silk suture, excised, placed in RNA later (Sigma-Aldrich, #R0901) and frozen at −80° C. 24 hours later. The heart was excised from each animal, and the right ventricle (RV) was carefully cut away from the left ventricle and septum (LV+S). Both pieces of heart tissue were separately weighed on a micro-balance (AJ000, Mettler) to measure the ratio of RV hypertrophy [RV/(LV+S); Fulton Index]. Lungs were perfused at 23 cm H2O with phosphate buffered solution (PBS, pH 7.4), then fixed with 10% neutral-buffered formalin (NBF) through cannulation of the pulmonary artery. Lungs were inflated with 10% NBF through cannulation of the trachea. Lungs remained in 10% NBF for 24 hours before being placed into 70% ethanol for at least 48 hours, before tissue processing and paraffin embedding. For lungs not perfusion fixed, the right inferior lobe was ligated with 5-0 silk suture before being excised, weighed and frozen in liquid N2.

### Results

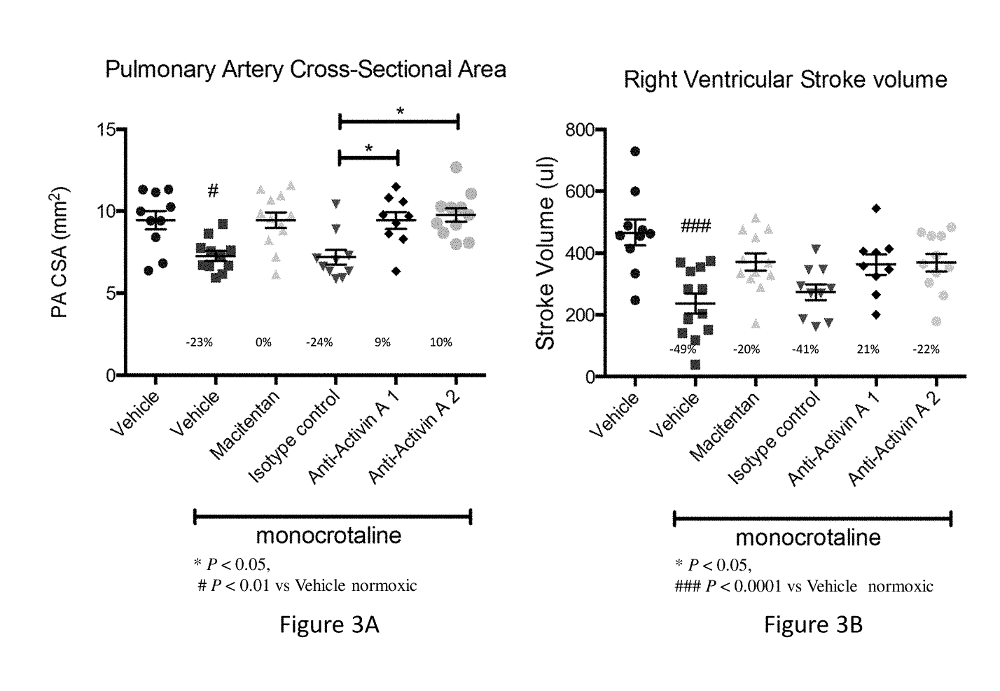

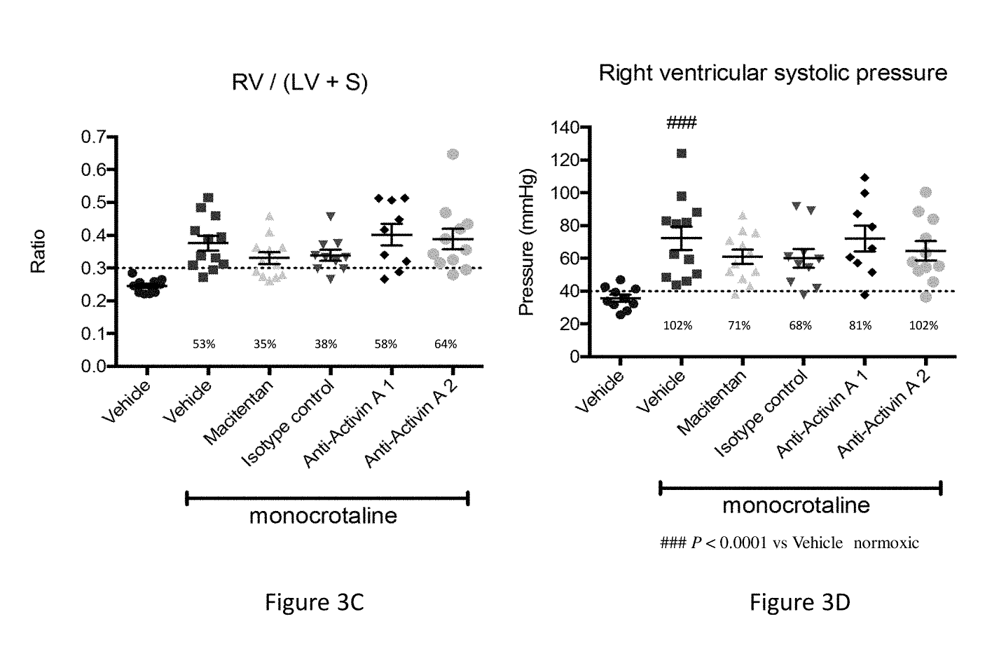

As shown in Table 7 and FIG. 3A, B-mode ultrasound imaging of the rat heart in cross-section revealed the pulmonary artery cross-sectional area (PA CSA) was significantly reduced in monocrotaline-injected rats orally dosed for 2 weeks with PEG 400. In other monocrotaline-injected rats, treatment with the isotype control antibody showed a similar response. Use of the endothelin receptor antagonist, Macitentan, restored PA CSA to that of saline-injected rats (not exposed to monocrotaline). Use of either of anti-Activin A antibodies, H4H10430P and H4H10446P2 restored PA CSA to values measured for saline-injected PEG 400 dosed rats.

TABLE 7
Average pulmonary artery cross-sectional area (PA CSA), stroke volume and
right ventricular cardiac output of treatment groups at end of study

| | | | | | Right |
|---|---|---|---|---|---|
| | | | | Stroke | Ventricular |
| | | | PA CSA | Volume | cardiac output |
| | | | (Ave ± SEM) | (Ave ± SEM) | (Ave ± SEM) |
| Group | Condition | Treatment | (mm²) | (uL) | (mL/min) |
| 1 | Saline | PEG 400 (50:50) | 9.450 ± 0.561 | 466.6 ± 41.6 | 167.9 ± 12.9 |
| 2 | Monocrotaline | PEG 400 (50:50) | 7.284 ± 0.303## | 237.2 ± 32.5#### | 85.4 ± 12.0#### |
| 3 | Monocrotaline | Macitentan | 9.458 ± 0.476** | 371.6 ± 27.9* | 127.8 ± 11.7 |
| 4 | Monocrotaline | PEG 400 (50:50) | 7.192 ± 0.453%% | 274.0 ± 25.9 | 98.9 ± 7.8 |
| | | Isotype control | | | |
| | | antibody | | | |
| 5 | Monocrotaline | PEG 400 (50:50) | 9.439 ± 0.516%% | 362.8 ± 32.6 | 130.0 ± 12.5 |
| | | H4H10430P | | | |
| 6 | Monocrotaline | PEG 400 (50:50) | 9.773 ± 0.410** | 369.0 ± 28.8 | 132.9 ± 12.6 |
| | | H4H10446P2 | | | |

ANOVA with Sidak's multiple comparisons:
##, ####P < 0.01, 0.0001 vs Saline-injected, PEG 400 dosed;
*, **P < 0.05, 0.01 vs Monocrotaline-injected, PEG 400 dosed;
%%P < 0.01 vs Monocrotaline-injected, REGN1945 dosed

Ultrasound M-mode imaging of the pulsed wave velocity time integral (VTI) found non-significant reductions in the velocity of blood flow through the pulmonary artery in animals exposed to monocrotaline. VTI and PA CSA were used to calculate the right ventricular stroke volume. Exposure to monocrotaline significantly reduced stroke volume in both PEG 400 and isotype control antibody treated rats. Anti-Activin A antibodies and macitentan partially restored stroke volume (FIG. 3B). Heart rate was measured and used to determine right ventricular cardiac output. In monocrotaline exposed rats, macitentan partially restored cardiac output in comparison to either PEG 400 or isotype control antibody treatment; anti-activin A antibodies showed similar restoration as Macitentan treatment yet were not statistically different from isotype control antibody.

Catheter-based assessment of heart rate and right ventricular pressures revealed no changes in heart rate, but significantly higher systolic pressures in rats exposed to monocrotaline (P<0.001 for Saline-injected vs monocrotaline-injected PEG 400 dosed rats). However, none of the treatment groups (Macitentan or anti-Activin A groups) showed attenuation in the elevation of right ventricular pressures (FIG. 3D). Assessment of ventricular contractility found higher dP/dt values in monocrotaline treated rats, indicating greater ventricular contractility to counteract the increased resistance in the pulmonary arteries likely a result of endothelial dysfunction caused by monocrotaline. However, drug treatment did not lower the pressures to values observed in rats injected with saline.

Post-mortem analysis of the rat heart weight found non-significant changes to overall heart weight. When heart weight was normalized to body weight, no statistical differences were detected. Right ventricular weight was higher with exposure to monocrotaline and remained statistical significant when normalized to total heart weight for the comparison of saline to monocrotaline exposed rats. Left ventricular weights were not significantly affected with monocrotaline exposure but the values trended lower when LV+S weights were normalized to total heart weight. The ratio of the right ventricular weight to the left ventricular plus septal weight provides a hypertrophy index (i.e., Fulton Index), and the groups exposed to monocrotaline had right ventricular hypertrophy (P<0.001 for Saline-injected vs monocrotaline-injected PEG 400 dosed rats). Treatment with Macitentan or either of the two anti-Activin A antibodies did not lessen right ventricular hypertrophy (FIG. 3C).

### Example 4

### Anti-Activin A Antibody Treatment Increases Survival Time in a Rat Model of Pulmonary Hypertension Induced by Monocrotaline

To evaluate the survival benefit of anti-Activin A antibody in a pulmonary arterial hypertension model, a long-term rat monocrotaline in vivo model was used.

The following materials and methods were used for this study.

### Materials and Methods

Six-to-seven week old Sprague Dawley rats were used. Rats were separated into treatment groups such that body weights were similar among different groups. Rats were subcutaneously administered either 40 mg/kg of monocrotaline or 5 mL/kg of saline at day 0. At 14 days post-injection, saline-injected rats were subcutaneously treated with saline at 5 mL/kg, twice a week for 5 weeks, unless they died prematurely.

The monocrotaline-injected rats were separated into 4 groups. One group (n=10) was orally administered macitentan at 30 mg/kg daily. A second group (n=10) was subcutaneously administered saline at 5 mL/kg twice a week. A third group (n=10) was subcutaneously treated with either an isotype control antibody, H4H6334P. A fourth group (n=10) was subcutaneously treated with the anti-Activin A antibody, H4H10446P2, at 40 mg/kg twice a week. These monocrotaline-treated rats were started on therapy at two weeks post-monocrotaline and continued for 5 weeks, unless the animal died prematurely. The experimental dosing and treatment protocols are shown in Table 8.

Animals were monitored twice a day for signs of morbidity or mortality. Based on literature searches, 5-8 weeks after monocrotaline injection is typically a time where morbidity and mortality are likely. In the interest of the animal's health, animals would be euthanized once they reached an “under-conditioned” criteria.

TABLE 8
Therapeutic dosing and treatment protocol for each
group of rats in long-term monocrotaline model

| | | | | Fre- | | “n” |
|---|---|---|---|---|---|---|
| Group | Condition | Treatment | Dosage | quency | Route | size |
| 1 | Saline | Saline | 5 mL/kg | 2x/week | SC | 6 |
| 2 | Monocrotaline | Saline | 5 mL/kg | 2x/week | SC | 10 |
| 3 | Monocrotaline | Macitentan | 30 mg/kg | Daily | PO | 10 |
| 4 | Monocrotaline | Isotype | 40 mg/kg | 2x/wk | SC | 10 |
| | | control | | | | |
| | | antibody | | | | |
| | | (H4H6334P) | | | | |
| 5 | Monocrotaline | Anti-Activin | 40 mg/kg | 2x/wk | SC | 10 |
| | | A antibody | | | | |
| | | (H4H10446P2; | | | | |
| | | REGN2477) | | | | |

### Results

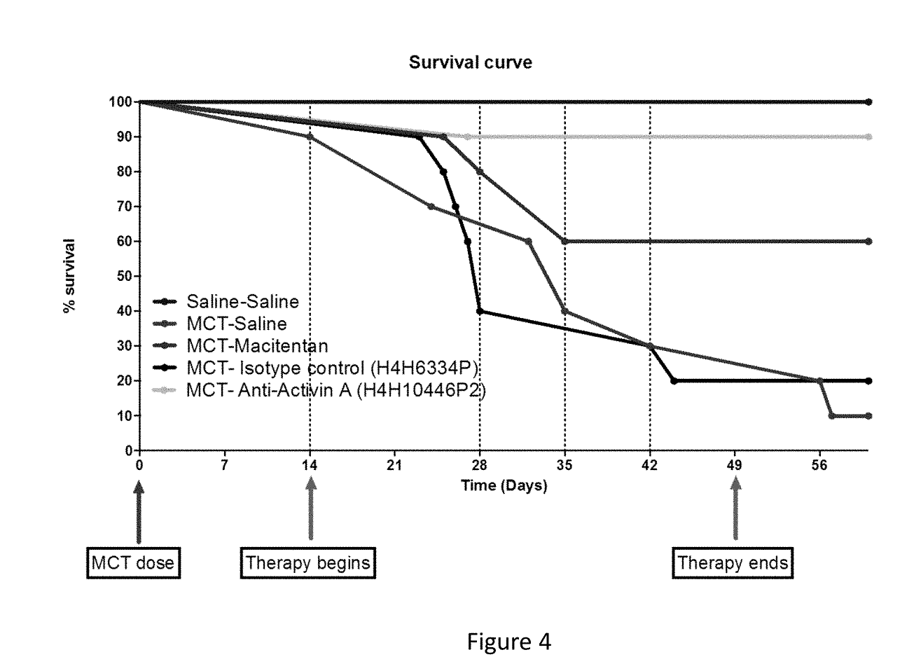

During the course of the study, 22 of 40 animals treated with 40 mg/kg of monocrotaline died (FIG. 4). Animal deaths occurred overnight, or during or after ultrasound image acquisition when animals were under anesthesia. The two groups of animals with the greatest mortality were the saline-treated animals and the isotype control antibody-treated animals with 90% and 80% mortality, respectively. Forty percent of the animals treated with Macitentan died but only 10% of the animals administered the anti-Activin A antibody, H4H10446P2, died. Accordingly treatment with an anti-Activin A antibody extends survival after monocrotaline injection.

### Equivalents

Those skilled in the art will recognize, or be able to ascertain using no more than routine experimentation, many equivalents to the specific embodiments and methods described herein. Such equivalents are intended to be encompassed by the scope of the following claims.

We claim:

1. A method of treating a subject having pulmonary arterial hypertension (PAH), comprising
administering to the subject a therapeutically effective amount of an anti-Activin A antibody, or antigen-binding fragment thereof,
wherein the therapeutic effect of administration of the anti-Activin A antibody, or antigen-binding fragment thereof, to the subject is selected from the group consisting of
inhibits thickening of the pulmonary artery in the subject;
increases stroke volume in the subject;
increases right ventricle cardiac output in the subject; and
extends survival time of the subject,
thereby treating the subject having PAH.

2. The method of claim 1, wherein the subject is human.

3. The method of claim 1, wherein the subject has Group I (WHO) PAH.

4. The method of claim 1, wherein the method further comprises administering to the subject at least one additional therapeutic agent.

5. The method of claim 4, wherein the therapeutic agent is selected from the group consisting of an anticoagulant, a diuretic, a cardiac glycoside, a calcium channel blocker, a vasodilator, a prostacyclin analogue, an endothelium antagonist, a phosphodiesterase inhibitor, an endopeptidase inhibitor, a lipid lowering agent, and a thromboxane inhibitor.

6. The method of claim 1, wherein the antibody, or antigen-binding fragment thereof, has a characteristic selected from the group consisting of
specifically binds Activin A with a binding dissociation equilibrium constant (K_{D}) of less than about 5 pM as measured in a surface plasmon resonance assay at 25° C.;
specifically binds Activin A with a binding dissociation equilibrium constant (K_{D}) of less than about 4 pM as measured in a surface plasmon resonance assay at 25° C.; and
specifically binds Activin A with a binding association equilibrium constant (Kₐ) of less than about 500 nM.

7. The method of claim 1, wherein the antibody, or antigen-binding fragment thereof, has a characteristic selected from the group consisting of
blocks binding of at least one Activin A receptor to Activin A;
blocks activation of at least one Activin A receptor by Activin A;
does not significantly block binding of Activin A to an Activin Type II receptor;
blocks Activin A binding to an Activin A receptor with an IC₅₀ value of less than about 80 pM as measured in an in vivo receptor/ligand binding bioassay at 25° C.;
blocks Activin A binding to an Activin A receptor with an IC₅₀ value of less than about 60 pM as measured in an in vivo receptor/ligand binding bioassay at 25° C.;
inhibits binding of Activin A to an Activin A receptor selected from the group consisting of Activin Type IIA receptor (ActRIIA), Activin Type IIB receptor (ActRIIB), and Activin Type I receptor; and
inhibits Activin A-mediated activation of SMAD complex signaling.

8. The method of claim 1, wherein the antibody, or antigen-binding fragment thereof, competes for binding to Activin A with a reference antibody comprising a heavy chain variable region (HCVR)/light chain variable region (LCVR) sequence pair selected from the group consisting of SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

9. The method of claim 1, wherein the antibody, or antigen-binding fragment thereof, binds to the same epitope on Activin A as a reference antibody comprising an HCVR/LCVR sequence pair selected from the group consisting of SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

10. The method of claim 1, wherein the antibody, or antigen-binding fragment thereof, comprises the heavy and light chain CDRs of a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

11. The method of claim 10, wherein the antibody, or antigen-binding fragment thereof, comprises HCDR1-HCDR2-HCDR3-LCDR1-LCDR2-LCDR3 domains, respectively, selected from the group consisting of: SEQ ID NOs: 4-6-8-12-14-16; 20-22-24-28-30-32; 36-38-40-44-46-48; 52-54-56-60-62-64; 68-70-72-76-78-80; 84-86-88-92-94-96; 100-102-104-92-94-96; 108-110-112-92-94-96; 116-118-120-92-94-96; 124-126-128-92-94-96; 132-134-136-92-94-96; 140-142-144-148-150-152; 156-158-160-148-150-152; 164-166-168-148-150-152; 172-174-176-148-150-152; 180-182-184-148-150-152; 188-190-192-148-150-152; 196-198-200-148-150-152; and 204-206-208-212-214-216.

12. The method of claim 1, wherein the antibody, or antigen-binding fragment thereof, comprises: (a) a HCVR having an amino acid sequence selected from the group consisting of SEQ ID NOs: 2, 18, 34, 50, 66, 82, 98, 106, 114, 122, 130, 138, 154, 162, 170, 178, 186, 194, and 202; and (b) a LCVR having an amino acid sequence selected from the group consisting of SEQ ID NOs: 10, 26, 42, 58, 74, 90, 146, and 210.

13. The method of claim 12, wherein the antibody or antigen-binding fragment thereof, comprises a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.


SEQUENCE LISTING (228 sequences)

SEQ ID NO: 1 - DNA, 366 bp, Artificial Sequence
  Synthetic
caggtacagctgcagcagtcaggtccaggactgctgaagccctcgcagaccctctcactcacctgtgccatctccggggacagtgtctctagcaacagtgctgcttggagttggatcaggcagtccccatcgagaggccttgagtggctgggaaggacatattacagggccaactggtttaatgattatgcactttctgtgaaaagtcgaataaccatcaacccagtcacatccacgaaccacttctccctgcagctgcactctgtgactcccgaggacacggctgtgtattactgtgcaagagaaggggctctgggatactactttgactcctggggccagggaaccctggtcaccgtctcctca

SEQ ID NO: 2 - protein, 122 aa, Artificial Sequence
  Synthetic
QVQLQQSGPGLLKPSQTLSLTCAISGDSVSSNSAAWSWIRQSPSRGLEWLGRTYYRANWFNDYALSVKSRITINPVTSTNHFSLQLHSVTPEDTAVYYCAREGALGYYFDSWGQGTLVTVSS

SEQ ID NO: 3 - DNA, 30 bp, Artificial Sequence
  Synthetic
ggggacagtgtctctagcaacagtgctgct

SEQ ID NO: 4 - protein, 10 aa, Artificial Sequence
  Synthetic
GDSVSSNSAA

SEQ ID NO: 5 - DNA, 27 bp, Artificial Sequence
  Synthetic
acatattacagggccaactggtttaat

SEQ ID NO: 6 - protein, 9 aa, Artificial Sequence
  Synthetic
TYYRANWFN

SEQ ID NO: 7 - DNA, 36 bp, Artificial Sequence
  Synthetic
gcaagagaaggggctctgggatactactttgactcc

SEQ ID NO: 8 - protein, 12 aa, Artificial Sequence
  Synthetic
AREGALGYYFDS

SEQ ID NO: 9 - DNA, 339 bp, Artificial Sequence
  Synthetic
gacatcgtgatgacccagtctccagactccctggctgtgtctctgggcgagagggccaccatcaactgcaagtccagtcaaagtgttttatacagctccaacaataagaattatttagcttggtaccaacagaaaccagggcagcctcctacactgctcatttactgggcatctacccgggaatccggggtccctgaccgattcagtggcagcgggtctgggacagatttcactctcaccatcagcagcctgcaggcggaagatgtggcaatttattactgtcaccaatattttattactccactcactttcggcggagggaccaaggtggagatcaaa

SEQ ID NO: 10 - protein, 113 aa, Artificial Sequence
  Synthetic
DIVMTQSPDSLAVSLGERATINCKSSQSVLYSSNNKNYLAWYQQKPGQPPTLLIYWASTRESGVPDRFSGSGSGTDFTLTISSLQAEDVAIYYCHQYFITPLTFGGGTKVEIK

SEQ ID NO: 11 - DNA, 36 bp, Artificial Sequence
  Synthetic
caaagtgttttatacagctccaacaataagaattat

SEQ ID NO: 12 - protein, 12 aa, Artificial Sequence
  Synthetic
QSVLYSSNNKNY

SEQ ID NO: 13 - DNA, 9 bp, Artificial Sequence
  Synthetic
tgggcatct

SEQ ID NO: 14 - protein, 3 aa, Artificial Sequence
  Synthetic
WAS

SEQ ID NO: 15 - DNA, 27 bp, Artificial Sequence
  Synthetic
caccaatattttattactccactcact

SEQ ID NO: 16 - protein, 9 aa, Artificial Sequence
  Synthetic
HQYFITPLT

SEQ ID NO: 17 - DNA, 378 bp, Artificial Sequence
  Synthetic
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatacaatgcagactccgtgaagggccgattcaccatctccagagacaattccaagaacacggtgtatctgcaaatgaacagcctgagagccgaggacacggctgtgtattactgtgcgagagcccggaattacgatattttgactggttattataacctcggtatggacgtctggggccaagggaccacggtcaccgtctcctca

SEQ ID NO: 18 - protein, 126 aa, Artificial Sequence
  Synthetic
QVQLVESGGGVVQPGRSLRLSCAASGFTFSSYGMHWVRQAPGKGLEWVAVIWYDGSNKYNADSVKGRFTISRDNSKNTVYLQMNSLRAEDTAVYYCARARNYDILTGYYNLGMDVWGQGTTVTVSS

SEQ ID NO: 19 - DNA, 24 bp, Artificial Sequence
  Synthetic
ggattcaccttcagtagctatggc

SEQ ID NO: 20 - protein, 8 aa, Artificial Sequence
  Synthetic
GFTFSSYG

SEQ ID NO: 21 - DNA, 24 bp, Artificial Sequence
  Synthetic
atatggtatgatggaagtaataaa

SEQ ID NO: 22 - protein, 8 aa, Artificial Sequence
  Synthetic
IWYDGSNK

SEQ ID NO: 23 - DNA, 57 bp, Artificial Sequence
  Synthetic
gcgagagcccggaattacgatattttgactggttattataacctcggtatggacgtc

SEQ ID NO: 24 - protein, 19 aa, Artificial Sequence
  Synthetic
ARARNYDILTGYYNLGMDV

SEQ ID NO: 25 - DNA, 321 bp, Artificial Sequence
  Synthetic
gacatccagatgacccagtctccatcctccctgtctgcatctgtaggagacagagtcaccatcacttgccgggcaagtcagggcattagaaatgatttaggctggtatcaacagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaa

SEQ ID NO: 26 - protein, 107 aa, Artificial Sequence
  Synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 27 - DNA, 18 bp, Artificial Sequence
  Synthetic
cagggcattagaaatgat

SEQ ID NO: 28 - protein, 6 aa, Artificial Sequence
  Synthetic
QGIRND

SEQ ID NO: 29 - DNA, 9 bp, Artificial Sequence
  Synthetic
gctgcatcc

SEQ ID NO: 30 - protein, 3 aa, Artificial Sequence
  Synthetic
AAS

SEQ ID NO: 31 - DNA, 27 bp, Artificial Sequence
  Synthetic
ctacagcataatagttacccgtacact

SEQ ID NO: 32 - protein, 9 aa, Artificial Sequence
  Synthetic
LQHNSYPYT

SEQ ID NO: 33 - DNA, 375 bp, Artificial Sequence
  Synthetic
gaagtgcagctggtggagtctgggggaaacttggtacagtctggcaggtccctgagactctcctgtacagcctctggattcgcctttgatgattttgccatgcactgggtccggcaagttccagggaagggcctggagtgggtctcaggtattagttggaatagtgataccatcggctatgcggactctgtgaagggccgattcaccatttccagagacaacgcccagaactccctgtttctgcaaatggacagtctgagagctgaggacacggccttgtattactgtgtaaaagatatggttcggggacttataggctactactactacggtatggacgtctggggccaagggaccacggtcaccgtctcctca

SEQ ID NO: 34 - protein, 125 aa, Artificial Sequence
  Synthetic
EVQLVESGGNLVQSGRSLRLSCTASGFAFDDFAMHWVRQVPGKGLEWVSGISWNSDTIGYADSVKGRFTISRDNAQNSLFLQMDSLRAEDTALYYCVKDMVRGLIGYYYYGMDVWGQGTTVTVSS

SEQ ID NO: 35 - DNA, 24 bp, Artificial Sequence
  Synthetic
ggattcgcctttgatgattttgcc

SEQ ID NO: 36 - protein, 8 aa, Artificial Sequence
  Synthetic
GFAFDDFA

SEQ ID NO: 37 - DNA, 24 bp, Artificial Sequence
  Synthetic
attagttggaatagtgataccatc

SEQ ID NO: 38 - protein, 8 aa, Artificial Sequence
  Synthetic
ISWNSDTI

SEQ ID NO: 39 - DNA, 54 bp, Artificial Sequence
  Synthetic
gtaaaagatatggttcggggacttataggctactactactacggtatggacgtc

SEQ ID NO: 40 - protein, 18 aa, Artificial Sequence
  Synthetic
VKDMVRGLIGYYYYGMDV

SEQ ID NO: 41 - DNA, 321 bp, Artificial Sequence
  Synthetic
gaaatagtgttgacgcagtctccagccatcctgtctttgtctccaggggaaagagccatcctctcctgcagggccagtcagagtatttacacctacttatcctggtaccaacagacacctggccgggctcccaggctcctcatctatgagacatccagcagggccactggcatcccagccaggttcattggcagtgggtctgggacagacttcactctcaccatcagtagcctagagcctgaagattttgcattttattactgtcagcaccgtagcgactggcctcccacttttggccaggggaccaagctggagatcaaa

SEQ ID NO: 42 - protein, 107 aa, Artificial Sequence
  Synthetic
EIVLTQSPAILSLSPGERAILSCRASQSIYTYLSWYQQTPGRAPRLLIYETSSRATGIPARFIGSGSGTDFTLTISSLEPEDFAFYYCQHRSDWPPTFGQGTKLEIK

SEQ ID NO: 43 - DNA, 18 bp, Artificial Sequence
  Synthetic
cagagtatttacacctac

SEQ ID NO: 44 - protein, 6 aa, Artificial Sequence
  Synthetic
QSIYTY

SEQ ID NO: 45 - DNA, 9 bp, Artificial Sequence
  Synthetic
gagacatcc

SEQ ID NO: 46 - protein, 3 aa, Artificial Sequence
  Synthetic
ETS

SEQ ID NO: 47 - DNA, 27 bp, Artificial Sequence
  Synthetic
cagcaccgtagcgactggcctcccact

SEQ ID NO: 48 - protein, 9 aa, Artificial Sequence
  Synthetic
QHRSDWPPT

SEQ ID NO: 49 - DNA, 351 bp, Artificial Sequence
  Synthetic
caggtgcagctggtggagtcggggggaggcgtggtccagcctgggaggtccctgagactctcctgtgtagcgtctggattcaccgtcagtagttatggcattcactgggtccgccaggctccaggcaagggactggagtgggtgtcagttatatggtatgatggaagaaataaagactatgcagactccgtgaagggccgattcaccatctccagagacaattccaagaacacggtgtatttggaaatgaaaggcctgagagccgaggacacggctctttattattgtgcgagagacaaaactggggattttgactcctggggccagggaaccctggtcaccgtctcctca

SEQ ID NO: 50 - protein, 117 aa, Artificial Sequence
  Synthetic
QVQLVESGGGVVQPGRSLRLSCVASGFTVSSYGIHWVRQAPGKGLEWVSVIWYDGRNKDYADSVKGRFTISRDNSKNTVYLEMKGLRAEDTALYYCARDKTGDFDSWGQGTLVTVSS

SEQ ID NO: 51 - DNA, 24 bp, Artificial Sequence
  Synthetic
ggattcaccgtcagtagttatggc

SEQ ID NO: 52 - protein, 8 aa, Artificial Sequence
  Synthetic
GFTVSSYG

SEQ ID NO: 53 - DNA, 24 bp, Artificial Sequence
  Synthetic
atatggtatgatggaagaaataaa

SEQ ID NO: 54 - protein, 8 aa, Artificial Sequence
  Synthetic
IWYDGRNK

SEQ ID NO: 55 - DNA, 30 bp, Artificial Sequence
  Synthetic
gcgagagacaaaactggggattttgactcc

SEQ ID NO: 56 - protein, 10 aa, Artificial Sequence
  Synthetic
ARDKTGDFDS

SEQ ID NO: 57 - DNA, 321 bp, Artificial Sequence
  Synthetic
gacatccagatgacccagtctccatcctccctgtctgcatctgtaggtgacagagtcaccatcacttgccgggcaagtcagaacattaacagctttttaagttggtatcagcagaaaccaggaaaagcccctaagttcctgatctatgatgcttccagtatacaaagtggggccccatcgaggttcagtggcagtggatctgggacagatttcactctcaccatcagtagtctgcaacctgaagattttgcaacttactactgtcaacagagttacagttccccgttcacttttggccaggggaccaagctggagatcaaa

SEQ ID NO: 58 - protein, 107 aa, Artificial Sequence
  Synthetic
DIQMTQSPSSLSASVGDRVTITCRASQNINSFLSWYQQKPGKAPKFLIYDASSIQSGAPSRFSGSGSGTDFTLTISSLQPEDFATYYCQQSYSSPFTFGQGTKLEIK

SEQ ID NO: 59 - DNA, 18 bp, Artificial Sequence
  Synthetic
cagaacattaacagcttt

SEQ ID NO: 60 - protein, 6 aa, Artificial Sequence
  Synthetic
QNINSF

SEQ ID NO: 61 - DNA, 9 bp, Artificial Sequence
  Synthetic
gatgcttcc

SEQ ID NO: 62 - protein, 3 aa, Artificial Sequence
  Synthetic
DAS

SEQ ID NO: 63 - DNA, 27 bp, Artificial Sequence
  Synthetic
caacagagttacagttccccgttcact

SEQ ID NO: 64 - protein, 9 aa, Artificial Sequence
  Synthetic
QQSYSSPFT

SEQ ID NO: 65 - DNA, 375 bp, Artificial Sequence
  Synthetic
gaagtgcagctggtggagtctgggggaggcttggtacagcctggcaggtccctgagactctcctgtaaagcctctggattcgcctttgatgatttcgccatgcactgggtccggcaagctccagggaagggcctggagtgggtctcaggtattgtttggaacagtggtgacataggctatgcggactctgtgaagggccgattcaccatctccagagacaacgccaagaactccctgtatctgcaactgaatagtctgagaactgaggacacggccttgtatttctgtgtaaaagatatggttcggggacttatgggcttcaactattacggtatggacgtctggggccaagggaccacggtcaccgtctcctca

SEQ ID NO: 66 - protein, 125 aa, Artificial Sequence
  Synthetic
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 67 - DNA, 24 bp, Artificial Sequence
  Synthetic
ggattcgcctttgatgatttcgcc

SEQ ID NO: 68 - protein, 8 aa, Artificial Sequence
  Synthetic
GFAFDDFA

SEQ ID NO: 69 - DNA, 24 bp, Artificial Sequence
  Synthetic
attgtttggaacagtggtgacata

SEQ ID NO: 70 - protein, 8 aa, Artificial Sequence
  Synthetic
IVWNSGDI

SEQ ID NO: 71 - DNA, 54 bp, Artificial Sequence
  Synthetic
gtaaaagatatggttcggggacttatgggcttcaactattacggtatggacgtc

SEQ ID NO: 72 - protein, 18 aa, Artificial Sequence
  Synthetic
VKDMVRGLMGFNYYGMDV

SEQ ID NO: 73 - DNA, 321 bp, Artificial Sequence
  Synthetic
gaaattgtgttgacgcagtctccagccaccctgtctttgtctccaggggaaagagccaccctctcctgcagggccagtcaaactattagtacttatttagtctggtaccgacagagacctggccaggctcccagtctcctcatttatgatgcatccaacagggccactgacatcccagccaggttcagtggcagtgggtctgggacagacttcactctcaccatcagcagccttgagcctgaagattttgcagtttattactgtcagcagcgtagcaactggccgatcaccttcggccaagggacacgactggagattaaa

SEQ ID NO: 74 - protein, 107 aa, Artificial Sequence
  Synthetic
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 75 - DNA, 18 bp, Artificial Sequence
  Synthetic
caaactattagtacttat

SEQ ID NO: 76 - protein, 6 aa, Artificial Sequence
  Synthetic
QTISTY

SEQ ID NO: 77 - DNA, 9 bp, Artificial Sequence
  Synthetic
gatgcatcc

SEQ ID NO: 78 - protein, 3 aa, Artificial Sequence
  Synthetic
DAS

SEQ ID NO: 79 - DNA, 27 bp, Artificial Sequence
  Synthetic
cagcagcgtagcaactggccgatcacc

SEQ ID NO: 80 - protein, 9 aa, Artificial Sequence
  Synthetic
QQRSNWPIT

SEQ ID NO: 81 - DNA, 369 bp, Artificial Sequence
  Synthetic
gaagtgcagctggtggagtctgggggaggcttggtacagcctggcaggtccctgacactctcctgtgcagtctctggattcacctttgatgatcatgccatgcactgggtccggcaagctccagggaagggcctggagtgggtctcaggtattagttggaatagtgtaagtataggctatgcggactctgtgaagggccgattcacgatctccagagacaacgccaagacctccctctatctgcaaatgaacagtctgagagttgacgacacggccttatattactgtgtgcaagatttaaacgatattttgactggttatcccctctttgacttttggggccagggaaccctggtcaccgtctcctca

SEQ ID NO: 82 - protein, 123 aa, Artificial Sequence
  Synthetic
EVQLVESGGGLVQPGRSLTLSCAVSGFTFDDHAMHWVRQAPGKGLEWVSGISWNSVSIGYADSVKGRFTISRDNAKTSLYLQMNSLRVDDTALYYCVQDLNDILTGYPLFDFWGQGTLVTVSS

SEQ ID NO: 83 - DNA, 24 bp, Artificial Sequence
  Synthetic
ggattcacctttgatgatcatgcc

SEQ ID NO: 84 - protein, 8 aa, Artificial Sequence
  Synthetic
GFTFDDHA

SEQ ID NO: 85 - DNA, 24 bp, Artificial Sequence
  Synthetic
attagttggaatagtgtaagtata

SEQ ID NO: 86 - protein, 8 aa, Artificial Sequence
  Synthetic
ISWNSVSI

SEQ ID NO: 87 - DNA, 48 bp, Artificial Sequence
  Synthetic
gtgcaagatttaaacgatattttgactggttatcccctctttgacttt

SEQ ID NO: 88 - protein, 16 aa, Artificial Sequence
  Synthetic
VQDLNDILTGYPLFDF

SEQ ID NO: 89 - DNA, 324 bp, Artificial Sequence
  Synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 90 - protein, 108 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 91 - DNA, 18 bp, Artificial Sequence
  Synthetic
cagagcattagcagctat

SEQ ID NO: 92 - protein, 6 aa, Artificial Sequence
  Synthetic
QSISSY

SEQ ID NO: 93 - DNA, 9 bp, Artificial Sequence
  Synthetic
gctgcatcc

SEQ ID NO: 94 - protein, 3 aa, Artificial Sequence
  Synthetic
AAS

SEQ ID NO: 95 - DNA, 30 bp, Artificial Sequence
  Synthetic
caacagagttacagtacccctccgatcacc

SEQ ID NO: 96 - protein, 10 aa, Artificial Sequence
  Synthetic
QQSYSTPPIT

SEQ ID NO: 97 - DNA, 369 bp, Artificial Sequence
  Synthetic
gaagtgcagctggtggagtctgggggaggcttggtacaggctggcaggtccctaagactctcctgtgaagcctctggattcacctttgatgattatggcatgcactgggtccggcaaggtccagggaagggcctggagtgggtctcaggtattagttggaatagtggtaacatagactatgcggactctgtgaagggccgattcaccatctccagagacaacgccaagacctccctgtatctgcaaatgaacagtctgaaaactgacgacacggccttgtatttctgtgcaaaagatgctgtggggtttaactggaactactttctctttgactactggggccagggaaccctggtcaccgtctcctca

SEQ ID NO: 98 - protein, 123 aa, Artificial Sequence
  Synthetic
EVQLVESGGGLVQAGRSLRLSCEASGFTFDDYGMHWVRQGPGKGLEWVSGISWNSGNIDYADSVKGRFTISRDNAKTSLYLQMNSLKTDDTALYFCAKDAVGFNWNYFLFDYWGQGTLVTVSS

SEQ ID NO: 99 - DNA, 24 bp, Artificial Sequence
  Synthetic
ggattcacctttgatgattatggc

SEQ ID NO: 100 - protein, 8 aa, Artificial Sequence
  Synthetic
GFTFDDYG

SEQ ID NO: 101 - DNA, 24 bp, Artificial Sequence
  Synthetic
attagttggaatagtggtaacata

SEQ ID NO: 102 - protein, 8 aa, Artificial Sequence
  Synthetic
ISWNSGNI

SEQ ID NO: 103 - DNA, 48 bp, Artificial Sequence
  Synthetic
gcaaaagatgctgtggggtttaactggaactactttctctttgactac

SEQ ID NO: 104 - protein, 16 aa, Artificial Sequence
  Synthetic
AKDAVGFNWNYFLFDY

SEQ ID NO: 105 - DNA, 351 bp, Artificial Sequence
  Synthetic
caggtgcagctggtggagtctgggggaggcgtggtccagcctgggaggtccctgagactcacctgtgtagcgtctggattcaccgtcagtagttatggaatgcactgggtccgccaggccccaggcaaggggctggagtgggtggcagttatgttttatgatgaaagtaaaaaatattatgcagactccgtgaagggccgattcaccatctccagagacaattccaagaacacgctgtatctgcaaatgaacagcctgagagtcgaggacacggctgtgtattactgtgcgagagatgaacagctcgactttgaatactggggccagggaaccctggtcaccgtctcctca

SEQ ID NO: 106 - protein, 117 aa, Artificial Sequence
  Synthetic
QVQLVESGGGVVQPGRSLRLTCVASGFTVSSYGMHWVRQAPGKGLEWVAVMFYDESKKYYADSVKGRFTISRDNSKNTLYLQMNSLRVEDTAVYYCARDEQLDFEYWGQGTLVTVSS

SEQ ID NO: 107 - DNA, 24 bp, Artificial Sequence
  Synthetic
ggattcaccgtcagtagttatgga

SEQ ID NO: 108 - protein, 8 aa, Artificial Sequence
  Synthetic
GFTVSSYG

SEQ ID NO: 109 - DNA, 24 bp, Artificial Sequence
  Synthetic
atgttttatgatgaaagtaaaaaa

SEQ ID NO: 110 - protein, 8 aa, Artificial Sequence
  Synthetic
MFYDESKK

SEQ ID NO: 111 - DNA, 30 bp, Artificial Sequence
  Synthetic
gcgagagatgaacagctcgactttgaatac

SEQ ID NO: 112 - protein, 10 aa, Artificial Sequence
  Synthetic
ARDEQLDFEY

SEQ ID NO: 113 - DNA, 369 bp, Artificial Sequence
  Synthetic
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatataatggggaactgggactacttctacggtatggacgtctggggccaagggaccacggtcaccgtctcctca

SEQ ID NO: 114 - protein, 123 aa, Artificial Sequence
  Synthetic
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIMGNWDYFYGMDVWGQGTTVTVSS

SEQ ID NO: 115 - DNA, 24 bp, Artificial Sequence
  Synthetic
ggattcacctttgatgattatgcc

SEQ ID NO: 116 - protein, 8 aa, Artificial Sequence
  Synthetic
GFTFDDYA

SEQ ID NO: 117 - DNA, 24 bp, Artificial Sequence
  Synthetic
attagttggaatagtggtagcata

SEQ ID NO: 118 - protein, 8 aa, Artificial Sequence
  Synthetic
ISWNSGSI

SEQ ID NO: 119 - DNA, 48 bp, Artificial Sequence
  Synthetic
gcaaaagatataatggggaactgggactacttctacggtatggacgtc

SEQ ID NO: 120 - protein, 16 aa, Artificial Sequence
  Synthetic
AKDIMGNWDYFYGMDV

SEQ ID NO: 121 - DNA, 369 bp, Artificial Sequence
  Synthetic
gaagtgcagctggtggagtctgggggaggcttggtacagcctggcaggtccctgagactctcctgtgcagcctctggattcacctttgatgataatgccatgcactgggtccggcaacctccagggaagggcctggagtgggtctcaggtattagttggaatagtggaagcataggctatgcggactctgtgaagggccgattcaccatctccagagacaacgccaagaactccctgtatctgcaaatgaacagtctgagagctgaggacacggccttgtattactgtgcaaaagatataaacgatattttgactggttatcctctttttgattactggggccagggaaccctggtcaccgtctcctca

SEQ ID NO: 122 - protein, 123 aa, Artificial Sequence
  Synthetic
EVQLVESGGGLVQPGRSLRLSCAASGFTFDDNAMHWVRQPPGKGLEWVSGISWNSGSIGYADSVKGRFTISRDNAKNSLYLQMNSLRAEDTALYYCAKDINDILTGYPLFDYWGQGTLVTVSS

SEQ ID NO: 123 - DNA, 24 bp, Artificial Sequence
  Synthetic
ggattcacctttgatgataatgcc

SEQ ID NO: 124 - protein, 8 aa, Artificial Sequence
  Synthetic
GFTFDDNA

SEQ ID NO: 125 - DNA, 24 bp, Artificial Sequence
  Synthetic
attagttggaatagtggaagcata

SEQ ID NO: 126 - protein, 8 aa, Artificial Sequence
  Synthetic
ISWNSGSI

SEQ ID NO: 127 - DNA, 48 bp, Artificial Sequence
  Synthetic
gcaaaagatataaacgatattttgactggttatcctctttttgattac

SEQ ID NO: 128 - protein, 16 aa, Artificial Sequence
  Synthetic
AKDINDILTGYPLFDY

SEQ ID NO: 129 - DNA, 378 bp, Artificial Sequence
  Synthetic
gaagtgcagctggtggagtctgggggaggcgtggtacagcctggggggtccctgagactctcctgtgcagcctctacattcacctttgatgattttgccatgcactgggtccgtcaagctccagggaagggtctggagtgggtctctcttattactggggatggtggtagcacatactatgcagaccctgtgaagggccgattcaccatctccagagacaacagcaaaaactccctgtatctgcaaatgaacagtctgagaactgaggacaccgccttgtattactgtgtaaaagattggtggatagcagctcgtccggactactactactacggtatggacgtctggggccaagggaccacggtcaccgtctcctca

SEQ ID NO: 130 - protein, 126 aa, Artificial Sequence
  Synthetic
EVQLVESGGGVVQPGGSLRLSCAASTFTFDDFAMHWVRQAPGKGLEWVSLITGDGGSTYYADPVKGRFTISRDNSKNSLYLQMNSLRTEDTALYYCVKDWWIAARPDYYYYGMDVWGQGTTVTVSS

SEQ ID NO: 131 - DNA, 24 bp, Artificial Sequence
  Synthetic
acattcacctttgatgattttgcc

SEQ ID NO: 132 - protein, 8 aa, Artificial Sequence
  Synthetic
TFTFDDFA

SEQ ID NO: 133 - DNA, 24 bp, Artificial Sequence
  Synthetic
attactggggatggtggtagcaca

SEQ ID NO: 134 - protein, 8 aa, Artificial Sequence
  Synthetic
ITGDGGST

SEQ ID NO: 135 - DNA, 57 bp, Artificial Sequence
  Synthetic
gtaaaagattggtggatagcagctcgtccggactactactactacggtatggacgtc

SEQ ID NO: 136 - protein, 19 aa, Artificial Sequence
  Synthetic
VKDWWIAARPDYYYYGMDV

SEQ ID NO: 137 - DNA, 378 bp, Artificial Sequence
  Synthetic
caggtgcagctgcaggagtcgggcccaggactggtgaagccctcggagaccctgtccatcacctgcactgtctctggtggctccttcagtagtcacttctggacctggatccggcagcccccaggaaagggactggaatggattggatatctccattatagtgggggcaccagctacaacccctccctcaagagtcgagtcatcatatcagtggacacgtccaagaaccagttctccctgaaactgaactctgtgaccgctgcggacacggccgtgtattactgtgcgagagctagatcggggattacttttgggggacttatcgtccctggttcttttgatatctggggccaagggacaatggtcaccgtctcttca

SEQ ID NO: 138 - protein, 126 aa, Artificial Sequence
  Synthetic
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSS

SEQ ID NO: 139 - DNA, 24 bp, Artificial Sequence
  Synthetic
ggtggctccttcagtagtcacttc

SEQ ID NO: 140 - protein, 8 aa, Artificial Sequence
  Synthetic
GGSFSSHF

SEQ ID NO: 141 - DNA, 21 bp, Artificial Sequence
  Synthetic
ctccattatagtgggggcacc

SEQ ID NO: 142 - protein, 7 aa, Artificial Sequence
  Synthetic
LHYSGGT

SEQ ID NO: 143 - DNA, 60 bp, Artificial Sequence
  Synthetic
gcgagagctagatcggggattacttttgggggacttatcgtccctggttcttttgatatc

SEQ ID NO: 144 - protein, 20 aa, Artificial Sequence
  Synthetic
ARARSGITFGGLIVPGSFDI

SEQ ID NO: 145 - DNA, 324 bp, Artificial Sequence
  Synthetic
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 146 - protein, 108 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 147 - DNA, 21 bp, Artificial Sequence
  Synthetic
cagtcagtctctagctcttat

SEQ ID NO: 148 - protein, 7 aa, Artificial Sequence
  Synthetic
QSVSSSY

SEQ ID NO: 149 - DNA, 9 bp, Artificial Sequence
  Synthetic
ggggcaagt

SEQ ID NO: 150 - protein, 3 aa, Artificial Sequence
  Synthetic
GAS

SEQ ID NO: 151 - DNA, 27 bp, Artificial Sequence
  Synthetic
caacagtacggaagcagcccgtggacg

SEQ ID NO: 152 - protein, 9 aa, Artificial Sequence
  Synthetic
QQYGSSPWT

SEQ ID NO: 153 - DNA, 378 bp, Artificial Sequence
  Synthetic
caggtgcagctgcaggagtcgggcccaggactggtgaagccttcggagaccctgtccctcatttgttctgtctctggtggctccttcagtagtcacttctggagttggatccggcagcccccagggaagggactggagtggattgggtatgtcctttacagtgggggcaccaattacaacccctccctcaagagtcgagtcaccatatcagtagacacgtccaagaatcagttcttcctgaaactgagctctgtgaccgctgcggacacggccgattattactgtgcgagagctatatcggggattacgtttgggggaattatcgtccctggttcttttgatatctggggccaagggacaatggtcaccgtctcttca

SEQ ID NO: 154 - protein, 126 aa, Artificial Sequence
  Synthetic
QVQLQESGPGLVKPSETLSLICSVSGGSFSSHFWSWIRQPPGKGLEWIGYVLYSGGTNYNPSLKSRVTISVDTSKNQFFLKLSSVTAADTADYYCARAISGITFGGIIVPGSFDIWGQGTMVTVSS

SEQ ID NO: 155 - DNA, 24 bp, Artificial Sequence
  Synthetic
ggtggctccttcagtagtcacttc

SEQ ID NO: 156 - protein, 8 aa, Artificial Sequence
  Synthetic
GGSFSSHF

SEQ ID NO: 157 - DNA, 21 bp, Artificial Sequence
  Synthetic
gtcctttacagtgggggcacc

SEQ ID NO: 158 - protein, 7 aa, Artificial Sequence
  Synthetic
VLYSGGT

SEQ ID NO: 159 - DNA, 60 bp, Artificial Sequence
  Synthetic
gcgagagctatatcggggattacgtttgggggaattatcgtccctggttcttttgatatc

SEQ ID NO: 160 - protein, 20 aa, Artificial Sequence
  Synthetic
ARAISGITFGGIIVPGSFDI

SEQ ID NO: 161 - DNA, 378 bp, Artificial Sequence
  Synthetic
caggtgcagctgcaggagtcgggcccaggactggtgaagccctcggagaccctgtccctcacctgcactgtctctggtggctccttcagtagtcacttctggagctggatccggcagcccccagggaagggactggagtggattggatatatcttatacactgggggcaccagcttcaacccctccctcaagagtcgagtctccatgtcagtgggcacgtccaagaaccagttctccctgaaattgagctctgtgaccgctgcggacacggccgtatattactgtgcgagagctagatcggggataacgtttacgggtattatcgtccctggctcttttgatatctggggccaagggacaatggtcaccgtctcttca

SEQ ID NO: 162 - protein, 126 aa, Artificial Sequence
  Synthetic
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 163 - DNA, 24 bp, Artificial Sequence
  Synthetic
ggtggctccttcagtagtcacttc

SEQ ID NO: 164 - protein, 8 aa, Artificial Sequence
  Synthetic
GGSFSSHF

SEQ ID NO: 165 - DNA, 21 bp, Artificial Sequence
  Synthetic
atcttatacactgggggcacc

SEQ ID NO: 166 - protein, 7 aa, Artificial Sequence
  Synthetic
ILYTGGT

SEQ ID NO: 167 - DNA, 60 bp, Artificial Sequence
  Synthetic
gcgagagctagatcggggataacgtttacgggtattatcgtccctggctcttttgatatc

SEQ ID NO: 168 - protein, 20 aa, Artificial Sequence
  Synthetic
ARARSGITFTGIIVPGSFDI

SEQ ID NO: 169 - DNA, 378 bp, Artificial Sequence
  Synthetic
caggtgcagctgcaggagtcgggcccaggactggtgaagccttcggagaccctgtccctcacttgttctgtctctggtggctccttcagtagtcacttctggagctggatccggcagcccccagggaagggactggagtggattggatatatccattacagtgggggcaccaattacaacccctccctcaagagtcgagtcaccatatcagtagacacgtccaagaaccagttctcccttaaactgacttctgtgaccgctgcggacacggccgattattactgtgcgagagctatatcggggattacgtttgggggaatgatcgtccctggttcttttgatgtctggggcgaagggacaatggtcaccgtctcttca

SEQ ID NO: 170 - protein, 126 aa, Artificial Sequence
  Synthetic
QVQLQESGPGLVKPSETLSLTCSVSGGSFSSHFWSWIRQPPGKGLEWIGYIHYSGGTNYNPSLKSRVTISVDTSKNQFSLKLTSVTAADTADYYCARAISGITFGGMIVPGSFDVWGEGTMVTVSS

SEQ ID NO: 171 - DNA, 24 bp, Artificial Sequence
  Synthetic
ggtggctccttcagtagtcacttc

SEQ ID NO: 172 - protein, 8 aa, Artificial Sequence
  Synthetic
GGSFSSHF

SEQ ID NO: 173 - DNA, 21 bp, Artificial Sequence
  Synthetic
atccattacagtgggggcacc

SEQ ID NO: 174 - protein, 7 aa, Artificial Sequence
  Synthetic
IHYSGGT

SEQ ID NO: 175 - DNA, 60 bp, Artificial Sequence
  Synthetic
gcgagagctatatcggggattacgtttgggggaatgatcgtccctggttcttttgatgtc

SEQ ID NO: 176 - protein, 20 aa, Artificial Sequence
  Synthetic
ARAISGITFGGMIVPGSFDV

SEQ ID NO: 177 - DNA, 378 bp, Artificial Sequence
  Synthetic
caggtgcagctgcaggagtcgggcccaggactggtgaagccttcggagaccctgtccctcacctgcactgtctctggtggcaccttcagtagtcacttctggagctggatccggcagcccccaggaaagggactggagtggattggatatatcttttacactgggggcaccaaccacaacccctccctcaagagtcgagtcaccatatcaatagacacgtccaagaaccagttctccctgaaactgacctctgtgaccgctgcggacacggccgtgtattactgtgcgagagctagatcggggattacgtttgggggagttatcgtccctggttcttttgatatctggggccaagggacaatggtcaccgtctcttca

SEQ ID NO: 178 - protein, 126 aa, Artificial Sequence
  Synthetic
QVQLQESGPGLVKPSETLSLTCTVSGGTFSSHFWSWIRQPPGKGLEWIGYIFYTGGTNHNPSLKSRVTISIDTSKNQFSLKLTSVTAADTAVYYCARARSGITFGGVIVPGSFDIWGQGTMVTVSS

SEQ ID NO: 179 - DNA, 24 bp, Artificial Sequence
  Synthetic
ggtggcaccttcagtagtcacttc

SEQ ID NO: 180 - protein, 8 aa, Artificial Sequence
  Synthetic
GGTFSSHF

SEQ ID NO: 181 - DNA, 21 bp, Artificial Sequence
  Synthetic
atcttttacactgggggcacc

SEQ ID NO: 182 - protein, 7 aa, Artificial Sequence
  Synthetic
IFYTGGT

SEQ ID NO: 183 - DNA, 60 bp, Artificial Sequence
  Synthetic
gcgagagctagatcggggattacgtttgggggagttatcgtccctggttcttttgatatc

SEQ ID NO: 184 - protein, 20 aa, Artificial Sequence
  Synthetic
ARARSGITFGGVIVPGSFDI

SEQ ID NO: 185 - DNA, 378 bp, Artificial Sequence
  Synthetic
caggtgcagctgcaggagtcgggcccaggactggtgaaaccttcggagaccctgtccctcacctgcactgtctctggtggctccttcagcagtcacttctggaactggatccggcagtccccagggaggggactggaatggattggatatatctattacagtgggggcaccaactataacccctccttcaagagtcgagtcaccatgtcagtagacacgtccaagaaccagttctccctgaaactgagctctgtgaccgctgcggacacggccgtgttttactgtgcgagagctagatcggggataacgtttgggggagttctcgtccctggttcttttgatatttggggccaagggacaatggtcaccgtctcttca

SEQ ID NO: 186 - protein, 126 aa, Artificial Sequence
  Synthetic
QVQLQESGPGLVKPSETLSLTCTVSGGSFSSHFWNWIRQSPGRGLEWIGYIYYSGGTNYNPSFKSRVTMSVDTSKNQFSLKLSSVTAADTAVFYCARARSGITFGGVLVPGSFDIWGQGTMVTVSS

SEQ ID NO: 187 - DNA, 24 bp, Artificial Sequence
  Synthetic
ggtggctccttcagcagtcacttc

SEQ ID NO: 188 - protein, 8 aa, Artificial Sequence
  Synthetic
GGSFSSHF

SEQ ID NO: 189 - DNA, 21 bp, Artificial Sequence
  Synthetic
atctattacagtgggggcacc

SEQ ID NO: 190 - protein, 7 aa, Artificial Sequence
  Synthetic
IYYSGGT

SEQ ID NO: 191 - DNA, 60 bp, Artificial Sequence
  Synthetic
gcgagagctagatcggggataacgtttgggggagttctcgtccctggttcttttgatatt

SEQ ID NO: 192 - protein, 20 aa, Artificial Sequence
  Synthetic
ARARSGITFGGVLVPGSFDI

SEQ ID NO: 193 - DNA, 378 bp, Artificial Sequence
  Synthetic
caggtgcagctgcaggagtcgggcccaggactggtgaagccttcggagaccctgtccctcacctgcactgtctctggtggctccttcagtagtcacttctggagctggatccggcagcccccaggaaagggactggagtggattgggtatatctattacagtgggggcacccactacaacccctccctcgagagtcgagtcaccatatcagtagacacgtccaagaaccagttctccctgaaactgaactctgtgaccgctgcggacacggccgtttattactgtgcgagagctagatcggggattacttttgggggacttatcgtccctggttcttttgatatctggggccaagggacaatggtcaccgtctcttca

SEQ ID NO: 194 - protein, 126 aa, Artificial Sequence
  Synthetic
QVQLQESGPGLVKPSETLSLTCTVSGGSFSSHFWSWIRQPPGKGLEWIGYIYYSGGTHYNPSLESRVTISVDTSKNQFSLKLNSVTAADTAVYYCARARSGITFGGLIVPGSFDIWGQGTMVTVSS

SEQ ID NO: 195 - DNA, 24 bp, Artificial Sequence
  Synthetic
ggtggctccttcagtagtcacttc

SEQ ID NO: 196 - protein, 8 aa, Artificial Sequence
  Synthetic
GGSFSSHF

SEQ ID NO: 197 - DNA, 21 bp, Artificial Sequence
  Synthetic
atctattacagtgggggcacc

SEQ ID NO: 198 - protein, 7 aa, Artificial Sequence
  Synthetic
IYYSGGT

SEQ ID NO: 199 - DNA, 60 bp, Artificial Sequence; identical to SEQ ID NO: 143
  Synthetic

SEQ ID NO: 200 - protein, 20 aa, Artificial Sequence
  Synthetic
ARARSGITFGGLIVPGSFDI

SEQ ID NO: 201 - DNA, 375 bp, Artificial Sequence
  Synthetic
cgggtgcaactggtgcagtctgggtctgaggtgaagaagcctggggcctcagtgaaggtctcctgcagggcttctggatacatcttcaccagttatgatatcaattgggtgcgacaggccactggacaagggcttgagtggatgggatggatgaaccctaataatggtaacacagcctatacacagaagttccagggcagagtcaccatgaccaggaacacctccataagcacagcctacatggagctgagcagcctgagatctgaggacacggccgtgtattactgtgcgagaaagggattactatggttcgggaagttattagggtacggtatggacgtctggggccaagggaccacggtcaccgtctcctca

SEQ ID NO: 202 - protein, 125 aa, Artificial Sequence
  Synthetic
RVQLVQSGSEVKKPGASVKVSCRASGYIFTSYDINWVRQATGQGLEWMGWMNPNNGNTAYTQKFQGRVTMTRNTSISTAYMELSSLRSEDTAVYYCARKGLLWFGKLLGYGMDVWGQGTTVTVSS

SEQ ID NO: 203 - DNA, 24 bp, Artificial Sequence
  Synthetic
ggatacatcttcaccagttatgat

SEQ ID NO: 204 - protein, 8 aa, Artificial Sequence
  Synthetic
GYIFTSYD

SEQ ID NO: 205 - DNA, 24 bp, Artificial Sequence
  Synthetic
atgaaccctaataatggtaacaca

SEQ ID NO: 206 - protein, 8 aa, Artificial Sequence
  Synthetic
MNPNNGNT

SEQ ID NO: 207 - DNA, 54 bp, Artificial Sequence
  Synthetic
gcgagaaagggattactatggttcgggaagttattagggtacggtatggacgtc

SEQ ID NO: 208 - protein, 18 aa, Artificial Sequence
  Synthetic
ARKGLLWFGKLLGYGMDV

SEQ ID NO: 209 - DNA, 324 bp, Artificial Sequence
  Synthetic
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 210 - protein, 108 aa, Artificial Sequence; identical to SEQ ID NO: 146
  Synthetic

SEQ ID NO: 211 - DNA, 21 bp, Artificial Sequence
  Synthetic
cagagtgttagcagcagctac

SEQ ID NO: 212 - protein, 7 aa, Artificial Sequence
  Synthetic
QSVSSSY

SEQ ID NO: 213 - DNA, 9 bp, Artificial Sequence
  Synthetic
ggtgcatcc

SEQ ID NO: 214 - protein, 3 aa, Artificial Sequence
  Synthetic
GAS

SEQ ID NO: 215 - DNA, 27 bp, Artificial Sequence
  Synthetic
cagcagtatggtagctcaccttggacg

SEQ ID NO: 216 - protein, 9 aa, Artificial Sequence
  Synthetic
QQYGSSPWT

SEQ ID NO: 217 - protein, 120 aa, Artificial Sequence
  Synthetic
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 218 - protein, 8 aa, Artificial Sequence
  Synthetic
GFTFSAYA

SEQ ID NO: 219 - protein, 8 aa, Artificial Sequence
  Synthetic
ISGSGGSA

SEQ ID NO: 220 - protein, 13 aa, Artificial Sequence
  Synthetic
AKDGAWKMSGLDV

SEQ ID NO: 221 - protein, 107 aa, Artificial Sequence
  Synthetic
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 222 - protein, 6 aa, Artificial Sequence
  Synthetic
QDISDY

SEQ ID NO: 223 - protein, 3 aa, Artificial Sequence
  Synthetic
TTS

SEQ ID NO: 224 - protein, 9 aa, Artificial Sequence
  Synthetic
QKYDSAPLT

SEQ ID NO: 225 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 226 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 227 - protein, 340 aa, Artificial Sequence
  Synthetic
EAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 407 aa, Homo Sapiens [sequence omitted]